(19) United States
(12) Patent Application Publication
(10) Pub. No.: US 2023/0399387 A1
(43) Pub. Date: Dec. 14, 2023

(54) ANTIBODIES FOR USE IN THE DETECTION AND TREATMENT OF HEART DISEASE

(71) Applicants: MUSC FOUNDATION FOR RESEARCH DEVELOPMENT, Charleston, SC (US); BOARD OF REGENTS, THE UNIVERSITY OF TEXAS SYSTEM, Austin, TX (US)

(72) Inventors: Federica DEL MONTE, Mount Pleasant, SC (US); Rakez KAYED, Galveston, TX (US)

(73) Assignees: MUSC FOUNDATION FOR RESEARCH DEVELOPMENT, Charleston, SC (US); BOARD OF REGENTS, THE UNIVERSITY OF TEXAS SYSTEM, Austin, TX (US)

(21) Appl. No.: 18/033,464

(22) PCT Filed: Nov. 5, 2021
(86) PCT No.: PCT/US2021/072256

§ 371 Date: Apr. 24, 2023

Related U.S. Application Data
(60) Provisional application No. 63/110,415, filed on Nov. 6, 2020.

(51) Int. Cl. C07K 16/18; G01N 33/68; A61P 9/00

(52) U.S. Cl. CPC C07K 16/18; G01N 33/6896; A61P 9/00; C07K 2317/31; G01N 2800/32

(57) ABSTRACT

The present disclosure is directed to method of diagnosing and treating heart disease using antibodies that bind to Cofilin, Aβ and/or Tau.

### PRIORITY CLAIM

This application claims benefit priority to Provisional Application U.S. Ser. No. 63/110,415, filed Nov. 6, 2020, the entire contents of which are hereby incorporated by reference.

### BACKGROUND

### 1. Field of the Disclosure

The present disclosure relates generally to the fields of medicine, disease, and cardiology. More particular, the disclosure relates to human antibodies binding to mis-folded proteins, including Cofilin, Aβ ant Tau, in heart disease and their use in detecting and treating the same.

### 2. Background

Alzheimer's disease (AD) is a disorder of protein folding defined by the presence of deposits of amyloid-beta (Aβ) in senile plaques, and hyper-phosphorylated microtubule-associated protein tau composing neurofibrillary tangles (NFTs) (Ballatore et al., 2007). First described in AD, aggregates of hyper-phosphorylated tau are linked to a wide spectrum of neurodegenerative disorders including Frontotemporal Dementia with Parkinsonism, Pick Disease, Corticobasal Degeneration, Chronic Traumatic Encephalopathy and Progressive Supranuclear Palsy, underlying the pathogenic importance of tau in a range of illnesses of the adulthood (Kovacs, 2018). Notably, all tauopathies described so far are brain disorders.

Recently, AD and heart failure (HF) have been mechanistically linked, not only through perfusion defects (Iadecola, 2004; Ruitenberg et al., 2005) or abnormal vascular reactivity (Iturria-Medina et al., 2016), but also via the pathogenic mechanism of misfolded protein accumulation (Diteepeng et al., 2021; Daniele et al., 2020) and the pathological hallmark of Aβ fibrillar and non-fibrillar soluble pre-amyloid-oligomers (PAO) (Troncone et al., 2016). Tau, instead, has been considered a brain specific protein despite MAPT mRNA is expressed in other organs (including the heart), that rodent proteomics (Drastichova et al., 2012) suggested that tau is also expressed in the myocardium, and tubulin (stabilized by tau to form microtubules) plays a critical role in almost every cell in the body. However, whether tau also takes part in the pathogenesis of HF, and by which mechanism(s) is unknown.

### SUMMARY

Thus, in accordance with the present disclosure, there is provided a method of detecting a misfolded protein in the heart tissue of a subject comprising (a) contacting a heart tissue sample from said subject with an antibody or antibody fragment that binds to Cofilin, Aβ or Tau; and (b) detecting Cofilin, Aβ or Tau in said sample by binding of said antibody or antibody fragment to a Cofilin, Aβ or Tau in said sample. The sample may be a tissue sample, such as a cardiac tissue sample. Detection may comprise ELISA, RIA, lateral flow assay or Western blot. The method may further comprise performing steps (a) and (b) a second time and determining a change in Cofilin, Aβ or Tau levels as compared to the first assay. The antibody fragment may be a recombinant scFv (single chain fragment variable) antibody, Fab fragment, F(ab′)₂ fragment, or Fv fragment.

In another embodiment, there is provided a method of treating a subject with heart disease characterized by misfolded proteins in cardiac tissue, or reducing the likelihood of the same, comprising delivering to said subject an antibody or antibody fragment that binds to Cofilin, Aβ or Tau. The antibody fragment may be a recombinant scFv (single chain fragment variable) antibody, Fab fragment, F(ab′)₂ fragment, or Fv fragment. The may be an IgG, or a recombinant IgG antibody or antibody fragment comprising an Fc portion mutated to alter (eliminate or enhance) FcR interactions, to increase half-life and/or increase therapeutic efficacy, such as a LALA, LALA-PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern. The antibody may be a chimeric antibody or a bispecific antibody. The antibody or antibody fragment may be administered multiple times, such as weekly, every other week, monthly, every other month, every three months or every six months. Delivering may comprise antibody or antibody fragment administration, or genetic delivery with an RNA or DNA sequence or vector encoding the antibody or antibody fragment.

In yet another embodiment, there is provided a vaccine formulation comprising one or more antibodies or antibody fragments that bind to one, two or all three of Cofilin, Aβ or Tau. The vaccine formulation may further comprise a second heart failure therapy. At least one of said antibody fragments may a recombinant scFv (single chain fragment variable) antibody, Fab fragment, F(ab′)₂ fragment, or Fv fragment. At least one of said antibodies may be a chimeric antibody or is bispecific antibody. At least one of said antibodies may be an IgG, or a recombinant IgG antibody or antibody fragment comprising an Fc portion mutated to alter (eliminate or enhance) FcR interactions, to increase half-life and/or increase therapeutic efficacy, such as a LALA, LALA-PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern. At least one of said antibodies or antibody fragments further comprises a cell penetrating peptide and/or is an intrabody. Also provide is a vaccine formulation comprising one or more expression vectors encoding a first antibody or antibody fragment that bind to Cofilin, Aβ or Tau. The vaccine may comprise a single vector encoding two or all three antibodies or fragments or may include two or three distinct vectors encoding two or all three antibodies or fragments. The expression vector(s) may be Sindbis virus or VEE vector(s). The vaccine formulation may be formulated for delivery by needle injection, jet injection, or electroporation. The vaccine formulation may further comprise one or more expression vectors encoding for a second antibody or antibody fragment that binds to Cofilin, Aβ or Tau, such as wherein the second antibody binds to a different protein than the first antibody.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-I: Human hearts express big-tau and tau p-Ser^{396/404}: SDS page of tau expression in myocardial tissue soluble (FIG. 1A) and insoluble (FIG. 1B) fractions of iDCM and age/sex/ethnicity matched control. (FIG. 1C) quantification of tau expression. SDS page of tau expression in myocardial tissue lysate from the soluble (FIG. 1D) and insoluble fraction (FIG. 1E) of AD and age/sex/ethnicity matched control. (FIG. 1F) quantification of tau expression; (FIGS. 1G-I) total tau quantification by ELISA using tau 5 and phosphor-tau using p-Ser^{396/404} antibodies. Data are presented as means±SEM. P-values have been calculated using the non-parametric two-tailed Mann-Whitney test for the SDS page. Statistical analysis for the ELISA was performed in GraphPad Prism (version 9.0.1) using a two-way ANOVA with Bonferroni multiple comparison.

FIGS. 2A-B: Oligomerized tau accumulates in myocardial tissue in iDCM and AD: (FIG. 2A) immunofluorescence images of total tau (Tau 5 in green) and tau-PAO (T22 in red) in control and iDCM myocardial tissue; (FIG. 2B) immunofluorescence images of total tau (Tau 5 in green) and tau-PAO (T22 in red) and merged images in control, iDCM and AD myocardial tissue.

FIGS. 3A-L: Phospho-tau Ser³⁹⁶ and p-Ser²⁶² in human iDCM heat and AD heart and brain. Tau hyperphosphorylation on Ser³⁹⁶ (FIG. 3A) and segregation in the aggregates (FIG. 3B) in human iDCM hearts, while no change occurs in AD hearts (FIGS. 3D-F). Quantification of tau p-Ser²⁶² in iDCM (FIG. 3G, FIG. 3H) or AD hearts (FIG. 3I, FIG. 3L). Data are presented as means±SEM. P-values have been calculated using the non-parametric two-tailed Mann-Whitney test for the SDS page.

FIGS. 4A-C: Oligomerized tau accumulates in tissue and isolated cardiomyocytes in iDCM and AD: (FIG. 4A) immunofluorescence images of tau-PAO in control, iDCM and AD myocardial tissue; (FIG. 4B) intracellular aggregation of tau-PAO in isolated cardiomyocytes. (FIG. 4C) Manders coefficient of colocalization of total tau and tau-PAO. The arrow indicates the site of measurement of the colocalization. Green: total tau, Red: tau-PAO, DAPI in blue: nuclei.

FIGS. 5A-U: Functional effect and mechanisms of tauopathy in the heart in hTau and C57Bl/6J mice. Representative M-Mode echocardiographic images (FIG. 5A); pulse-wave doppler of the mitral valve (MV) flow (FIG. 5B) tissue doppler at the level of the mitral valve anulus (FIG. 5C). Systolic function parameter: fractional shortening (FS) (FIG. 5D). Diastolic function parameters: Left Atria (LA) dimensions (FIG. 5E); LA calculated pressure (FIG. 5F); MV E/A ratio (FIG. 5G); MV deceleration time (FIG. 5H); E/e′ ratio (FIG. 5I); (FIG. 5J) Index of right ventricular function: Right ventricular outflow tract (RVOT). Data are presented with dotted lines since the data were not collected longitudinally from the same mice. The appropriate representation as box-plot is shown in Fig. S2. (FIG. 5K) representative 2D image of the hTau heart in short axis showing paradoxical septal movement. Immunostaining of tau-PAO using TOMA antibodies (green) in: myocardial tissue of all three age groups (FIG. 5L) and quantitative analysis of the signal (FIG. 5M); and in isolated cardiomyocytes (FIG. 5N) and quantitative analysis of the signal (FIG. 5O). (FIGS. 5P-T) Cardiomyocyte function parameters; FIG. 5U) immunostaining of microtubules (red) and Ca²′ handling protein Ryanodine Receptors (RyRs) (green) in 12 months old mice. Data are presented as untransformed means±SEM. P-values have been calculated using two-tailed parametric t-test or nonparametric Mann Whitney U test. Cardiomyocytes P-values have been calculated using linear mixed-effects model (LMM) with post-hoc comparison with Holm-Bonferroni correction therefore to bar graph is not included. Data are graphed as the means of the groups based on the pooled average of the mice. represents the P-value obtained following log 10 transformation and LMM analysis.

FIGS. 6A-J: Structural and Functional myocardial outcomes of TOMA Immunotherapy for hTau mice. (FIG. 6A) Injection and readout measurements protocol; (FIGS. 6B-D) diastolic function parameters: E/e′ ratio (FIG. 6B); Left Atrium (LA) dimension (FIG. 6C); Mean Left Atrial Pressure (FIG. 6D). Representative M-Mode echocardiographic images (FIG. 6E); pulse-wave doppler of the mitral valve flow (FIG. 6F) tissue doppler at the level of the mitral valve annulus (FIG. 6G) of IgG or TOMA treated 12 months old hTau mice one month after injection. Immunohistochemistry of the IgG and TOMA treated mice to visualize: FIG. 6H) total tau (tau5 antibody) (blue), tau-PAO (T22 antibody) (red); WGA (green) defines the cell membrane to localize the intra/extracellular accumulation of oligomers; (FIG. 6J) polymerized Tubulin (red) and RyRs (green). (FIG. 6I) quantification of the oligomers. Data are presented as means±SEM. P-values have been calculated using two-tailed parametric t-test or non-parametric Mann-Whitney test.

SFIGS. 1A-E. Structural and cognitive function in hTau mice. (SFIGS. 1A-B) Morris water maze. Escape latency in the acquisition trials (SFIG. 1A); trajectories and time spent in the target quadrant in the probe trial (SFIG. 1B); (SFIG. 1C) Novelty Y-maze test. Trajectories and time spent in novel arm in the test session; (SFIG. 1D) Open field test. Trajectories and time spent in Periphery and Center in open field test; (SFIG. 1E) Immunostaining for oligomeric tau in the Prefrontal Cortex in C57Bl/6J (wild-type, WT) and hTau mice at 3-, 7-, and 12-months of age. C57Bl/6J wild-type (WT) mice: 6 males at 3 months, 4 male/2 female at 7 months and 8 male/1 female at 12 months of age; hTau mice: 6 Male at 3 months, 6 males at 7 months and 3 male/4 female at 12 months of age. The data are presented as means±SEM. The P-values have been calculated using two-way ANOVA with Bonferroni correction or non-parametric two-tailed Mann-Whitney test. Insert image of the brain was generated using BioRender.

SFIG. 2. Box plot of the echocardiographic parameters. Echocardiographic parameters in the 3-, 7- and 12-month-old mice represented as box plot since they were not the same mice followed longitudinally. Values are represented as mean+25th and 75th percentile.

The whiskers extend to the minimum and maximum values. Middle line is the median. WT=C57Bl/6J wild-type mice. Abbreviations: E/e′=early mitral inflow velocity to mitral annular early diastolic velocity ratio; RVOT VTI=Right Ventricular outflow tract velocity time integral.

SFIG. 3. Tau-PAO accumulation in WT and hTau mice. Left panel) Cumulative analysis of Tau-PAO aggregates in the heart and statistical difference. Right panels) Representative images of WT and hTau mice at 3, 7, 12 months of age in the reconstructed stitched images. Heart image stitching has been performed with BZ-X Analyzer Software. Single images have been capture using Objective PlanApo_1 10×0.45/4.00 mm (equipped in Keyence Microscope BZ-X 800E) in capturing mode monochrome 8 bit. Image size 1920×1440. Correct shading auto mode and compressed method has been used, overlapping grade 30%. And automatic adjust image position mode using pattern matching. Drawing of the heart was made using BioRender.

SFIGS. 4A-B. Images of diastolic dysfunction pattern in hTau mice. (SFIG. 4A) Representative images of hTau mice mitral valve (MV) flow at the four grades of diastolic dysfunction. Inserts show a diagram of the position of the recording of the flow and the analysis of the parameters of the MV flow (top and middle panel on the right) and the tissue doppler velocity (bottom right panel). (SFIG.4B) movie depicting the effect of elevated right ventricular pressure causing dyssynchronous movement of the interventricular septum. LA=Left Atrium; LV=Left Ventricle IVRT=isovolumetric relaxation time; DT=Deceleration time; E=blood flow peak velocity from left ventricular relaxation in early diastole; A=atrial contraction flow peak velocity in late diastole; E=early mitral inflow velocity; e′ mitral annular early diastolic velocity ratio.

SFIG. 5. Cardiomyocytes function parameters in individual C57Bl/6J WT and hTau mice. Values of sarcomeric relaxation and Ca2+ transient parameters in the individual mice. Each data point represents the cells measured from each mouse. The variability of the values may reflect the different degree of accumulation of tau-PAO in the cell population. WT=C57Bl/6J wild-type mice.

SFIG. 6. Images of Tau-PAO in isolated cardiomyocytes. Immunofluorescence high magnification images of isolated cardiomyocytes from 6-month-old hTau mice showing heterogeneous accumulation of Tau-PAO stained with TOMA (in green) in cardiomyocytes. α-sarcomeric actin counterstain (in red) for sarcomeric proteins. DAPI in blue stains the nucleus. Scale bar 10 μm. WT=C57Bl/6J wild-type mice.

### DESCRIPTION OF ILLUSTRATIVE EMBODIMENTS

As discussed above, heart disease is a major health concern both in the United States and around the world. One important facet of heart disease is Ca²⁺ dysregulation. The toxic effect of Aβ-oligomers in the heart has been published in Circulation 121(10): 1216-26 (2010) and the presence of Aβ in the heart of patients with Alzheimer's disease has been published in JACC 68:2395-407 (2016). The presence of Cofilin in the heart has been published in JACC 65 (12):1199-214 (2015).

At present, there currently exist no marketed therapeutics that target this specific pathological cascade. Here, the inventors propose that the general oligomer antibody (A11), the Aβ-specific oligomers (VIA) and the Tau oligomer monoclonal antibody (TOMA), all proven efficacious in the amelioration of neuronal dysfunction and symptomology in mouse models of tauopathy, can be beneficial for the treatment of patients with cardiomyopathy and/or heart failure. Notably, treatment approaches to heart failure with preserved or reduced ejection fraction has remained fairly stagnant over the past few decades. As current heart failure therapeutics target fluid retention and cardiovascular dynamics through modulation of the renin-angiotensin system and cardiac receptors, there exists a gap in treatment for those individuals whose disease process is mediated by the accumulation and disruption of misfolded proteins within the myocardium.

The inventors show here that misfolding of certain proteins can be at the origin of cardiomyopathies and heart failure. Mounting evidence from other groups supports this hypothesis. These proteins (namely cofilin, Aβ and Tau) are the same proteins that misfold and accumulate in the brain of patients with Alzheimer's disease, and accumulate in the heart of patients with cardiomyopathies and heart failure. The heart of patients with Alzheimer's disease is also affected by the same deposits of cofilin, Aβ as well as Tau protein. Small soluble fragments (oligomers) of the Amyloid Precursor Protein (APP)-Aβ as well as Tau protein exert toxic effect on cardiomyocytes by altering calcium homeostasis.

In particular, the inventors provide new evidence that the high-molecular-weight (HMW) isoform, also known as big-tau (Georgieff et al., 1993), pathogenic hyperphosphorylated tau, and toxic tau-PAO are detectable in the human heart and participate in the pathogenesis of HF with preserved ejection fraction (HFpEF). They linked tau pathology to HF in a humanized tau mouse model (hTau) and mechanistically connect cardiac tauopathy to tau-PAO and microtubules and Ca²′ handling proteins disarray, the last two being well-known abnormalities in HF (Bers, 2000; 2001; del Monte et al., 2002). Finally, the last two decades have seen a surge in the number of clinical trials of drug therapies for AD, identifying PAO disaggregation as a more successful target to reduce brain pathology and improve clinical symptoms (Sevigny et al., 2016; Bernini et al., 2016; Catillo-Carranza et al., 2015). These results support that targeting tau-PAO offers a potential new approach to treat Cardiac Alzheimer. Tau, instead, has been considered a brain specific protein despite MAPT mRNA is expressed in other organs (including the heart), that rodent proteomics (Drastichova et al., 2017) suggested that tau is also expressed in the myocardium, and tubulin (stabilized by tau to form microtubules) plays a critical role in almost every cell in the body. However, whether tau also takes part in the pathogenesis of HF, and by which mechanism(s) is unknown.

As shown here, misfolded cofilin, Aβ and Tau can induce cardiac dysfunction consistent with cardiomyopathy. Application of antibodies such as A11, VIA and TOMA can help diagnose and treat such this disease. These and other aspects of the disclosure are described in detail below.

### I. HEART DISEASE

Heart failure is one of the leading causes of morbidity and mortality in the world. In the U.S. alone, estimates indicate that 3 million people are currently living with cardiomyopathy, and another 400,000 are diagnosed on a yearly basis. Dilated cardiomyopathy (DCM), also referred to as “congestive cardiomyopathy,” is the most common form of the cardiomyopathies and has an estimated prevalence of nearly 40 per 100,000 individuals (Durand et al., 1995). Although there are other causes of DCM, familial dilated cardiomyopathy has been indicated as representing approximately 20% of “idiopathic” DCM. Approximately half of the DCM cases are idiopathic, with the remainder being associated with known disease processes. For example, serious myocardial damage can result from certain drugs used in cancer chemotherapy (e.g., doxorubicin and daunorubucin). In addition, many DCM patients are chronic alcoholics. Fortunately, for these patients, the progression of myocardial dysfunction may be stopped or reversed if alcohol consumption is reduced or stopped early in the course of disease. Peripartum cardiomyopathy is another idiopathic form of DCM, as is disease associated with infectious sequelae. In sum, cardiomyopathies, including DCM, are significant public health problems.

Heart disease and its manifestations, including coronary artery disease, myocardial infarction, congestive heart failure and cardiac hypertrophy, clearly presents a major health risk in the United States today. The cost to diagnose, treat and support patients suffering from these diseases is well into the billions of dollars. Two particularly severe manifestations of heart disease are myocardial infarction and cardiac hypertrophy.

With respect to myocardial infarction, typically an acute thrombocytic coronary occlusion occurs in a coronary artery as a result of atherosclerosis and causes myocardial cell death. Because cardiomyocytes are terminally differentiated and generally incapable of cell division, they are generally replaced by scar tissue when they die during the course of an acute myocardial infarction. Scar tissue is not contractile, fails to contribute to cardiac function, and often plays a detrimental role in heart function by expanding during cardiac contraction, or by increasing the size and effective radius of the ventricle, for example, becoming hypertrophic.

With respect to cardiac hypertrophy, one theory regards this as a disease that resembles aberrant development and, as such, raises the question of whether developmental signals in the heart can contribute to hypertrophic disease. Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to DCM, heart failure, and sudden death. In the United States, approximately half a million individuals are diagnosed with heart failure each year, with a mortality rate approaching 50%.

Diagnosis of dilated cardiomyopathy typically depends upon the demonstration of enlarged heart chambers, particularly enlarged ventricles. Enlargement is commonly observable on chest X-rays but is more accurately assessed using echocardiograms. DCM is often difficult to distinguish from acute myocarditis, valvular heart disease, coronary artery disease, and hypertensive heart disease. Once the diagnosis of dilated cardiomyopathy is made, every effort is made to identify and treat potentially reversible causes and prevent further heart damage. For example, coronary artery disease and valvular heart disease must be ruled out. Anemia, abnormal tachycardias, nutritional deficiencies, alcoholism, thyroid disease and/or other problems need to be addressed and controlled.

Treatment with pharmacological agents still represents the primary mechanism for reducing or eliminating the manifestations of heart failure. Diuretics constitute the first line of treatment for mild-to-moderate heart failure. Unfortunately, many of the commonly used diuretics (e.g., the thiazides) have numerous adverse effects. For example, certain diuretics may increase serum cholesterol and triglycerides. Moreover, diuretics are generally ineffective for patients suffering from severe heart failure. If diuretics are ineffective, vasodilatory agents may be used; the angiotensin converting (ACE) inhibitors (e.g., enalopril and lisinopril) not only provide symptomatic relief, they also have been reported to decrease mortality. Again, however, the ACE inhibitors are associated with adverse effects that result in their being contraindicated in patients with certain disease states (e.g., renal artery stenosis). Similarly, inotropic agent therapy (i.e., a drug that improves cardiac output by increasing the force of myocardial muscle contraction) is associated with a panoply of adverse reactions, including gastrointestinal problems and central nervous system dysfunction.

Thus, the currently used pharmacological agents have severe shortcomings in particular patient populations. The availability of new, safe and effective agents would undoubtedly benefit patients who either cannot use the pharmacological modalities presently available, or who do not receive adequate relief from those modalities. The prognosis for patients with DCM is variable, and depends upon the degree of ventricular dysfunction, with the majority of deaths occurring within five years of diagnosis. The inventors describe herein a novel therapeutic composition and methods for treating pathologic cardiac hypertrophy and heart failure.

A. Aβ

Amyloid beta (Aβ or Abeta) denotes peptides of 36-43 amino acids that are the main component of the amyloid plaques found in the brains of people with Alzheimer's disease. The peptides derive from the amyloid precursor protein (APP), which is cleaved by beta secretase and gamma secretase to yield Aβ. Aβ molecules can aggregate to form flexible soluble oligomers which may exist in several forms. It is now believed that certain misfolded oligomers (known as “seeds”) can induce other Aβ molecules to also take the misfolded oligomeric form, leading to a chain reaction akin to a prion infection. The oligomers are toxic to nerve cells. The other protein implicated in Alzheimer's disease, Tau protein, also forms such prion-like misfolded oligomers, and there is some evidence that misfolded Aβ can induce Tau to misfold. A study has suggested that APP and its amyloid potential is of ancient origins, dating as far back as early deuterostomes.

The normal function of Aβ is not well understood. Though some animal studies have shown that the absence of Aβ does not lead to any obvious loss of physiological function, several potential activities have been discovered for Aβ, including activation of kinase enzymes, protection against oxidative stress, regulation of cholesterol transport, functioning as a transcription factor, and anti-microbial activity (potentially associated with Aβ's pro-inflammatory activity).

The glymphatic system clears metabolic waste from the mammalian brain, and in particular beta amyloids. Indeed, a number of proteases have been implicated by both genetic and biochemical studies as being responsible for the recognition and degradation of beta amyloids; these include insulin degrading enzyme. and presequence protease The rate of removal is significantly increased during sleep. However, the significance of the lymphatic system in Aβ clearance in Alzheimer's disease is unknown.

Aβ is the main component of amyloid plaques, extracellular deposits found in the brains of people with Alzheimer's disease). Similar plaques appear in some variants of Lewy body dementia and in inclusion body myositis (a muscle disease), while Aβ can also form the aggregates that coat cerebral blood vessels in cerebral amyloid angiopathy. The plaques are composed of a tangle of regularly ordered fibrillar aggregates called amyloid fibers, a protein fold shared by other peptides such as the prions associated with protein misfolding diseases. As mentioned above, The toxic effect of Aβ-oligomers in the heart was reported in 2010 and the presence of Aβ in the heart of patients with Alzheimer's disease was reported in 2016.

Research suggests that soluble oligomeric forms of the peptide may be causative agents in the development of Alzheimer's disease. It is generally believed that Aβ oligomers are the most toxic. The ion channel hypothesis postulates that oligomers of soluble, non-fibrillar Aβ form membrane ion channels allowing the unregulated calcium influx into neurons that underlies disrupted calcium ion homeostasis and apoptosis seen in Alzheimer's disease. Computational studies have demonstrated that also Aβ peptides embedded into the membrane as monomers with predominant helical configuration, can oligomerize and eventually form channels whose stability and conformation are sensitively correlated to the concomitant presence and arrangement of cholesterol. A number of genetic, cell biology, biochemical and animal studies support the concept that Aβ plays a central role in the development of Alzheimer's disease pathology.

Brain Aβ is elevated in people with sporadic Alzheimer's disease. Aβ is the main constituent of brain parenchymal and vascular amyloid; it contributes to cerebrovascular lesions and is neurotoxic. It is unresolved how Aβ accumulates in the central nervous system and subsequently initiates the disease of cells. Some researchers have found that the Aβ oligomers induce some of the symptoms of Alzheimer's disease by competing with insulin for binding sites on the insulin receptor, thus impairing glucose metabolism in the brain. Significant efforts have been focused on the mechanisms responsible for Aβ production, including the proteolytic enzymes γ- and β-secretases which generate Aβ from its precursor protein, APP (amyloid precursor protein). Aβ circulates in plasma, cerebrospinal fluid (CSF) and brain interstitial fluid (ISF) mainly as soluble Aβ40 Senile plaques contain both Aβ40 and Aβ42, while vascular amyloid is predominantly the shorter Aβ40. Several sequences of Aβ were found in both lesions. Generation of Aβ in the central nervous system may take place in the neuronal axonal membranes after APP-mediated axonal transport of β-secretase and presenilin-1.

Increases in either total Aβ levels or the relative concentration of both Aβ40 and Aβ42 (where the former is more concentrated in cerebrovascular plaques and the latter in neuritic plaques) have been implicated in the pathogenesis of both familial and sporadic Alzheimer's disease. Due to its more hydrophobic nature, the Aβ42 is the most amyloidogenic form of the peptide. However the central sequence KLVFFAE is known to form amyloid on its own, and probably forms the core of the fibril. One study further correlated Aβ42 levels in the brain not only with onset of Alzheimer's disease, but also reduced cerebrospinal fluid pressure, suggesting that a build-up or inability to clear Aβ42 fragments may play a role into the pathology.

The “amyloid hypothesis,” that the plaques are responsible for the pathology of Alzheimer's disease, is accepted by the majority of researchers but is not conclusively established. An alternative hypothesis is that amyloid oligomers rather than plaques are responsible for the disease. Mice that are genetically engineered to express oligomers but not plaques (APP^{E693Q}) develop the disease. Furthermore, mice that are in addition engineered to convert oligomers into plaques (APP^{E693Q} X PS1ΔE9), are no more impaired than the oligomer only mice. Intra-cellular deposits of Tau protein are also seen in the disease, and may also be implicated, as has aggregation of alpha synuclein.

While Aβ has been implicated in cancer development, prompting studies on a variety of cancers to elucidate the nature of its possible effects, results are largely inconclusive. Aβ levels have been assessed in relation to a number of cancers, including esophageal, colorectal, lung, and hepatic, in response to observed reductions in risk for developing Alzheimer's disease in survivors of these cancers. All cancers were shown to be associated positively with increased Aβ levels, particularly hepatic cancers. This direction of association however has not yet been established. Studies focusing on human breast cancer cell lines have further demonstrated that these cancerous cells display an increased level of expression of amyloid precursor protein.

Adults with Down syndrome had accumulation of amyloid in association with evidence of Alzheimer's disease, including declines in cognitive functioning, memory, fine motor movements, executive functioning, and visuospatial skills.

Aβ is formed after sequential cleavage of the amyloid precursor protein (APP), a transmembrane glycoprotein of undetermined function. APP can be cleaved by the proteolytic enzymes α-, β- and γ-secretase; Aβ protein is generated by successive action of the β and γ secretases. The γ secretase, which produces the C-terminal end of the Aβ peptide, cleaves within the transmembrane region of APP and can generate a number of isoforms of 30-51 amino acid residues in length. The most common isoforms are Aβ₄₀ and Aβ₄₂; the longer form is typically produced by cleavage that occurs in the endoplasmic reticulum, while the shorter form is produced by cleavage in the trans-Golgi network.

Autosomal-dominant mutations in APP cause hereditary early-onset Alzheimer's disease (familial AD). This form of AD accounts for no more than 10% of all cases, and the vast majority of AD is not accompanied by such mutations. However, familial Alzheimer's disease is likely to result from altered proteolytic processing. The gene for the amyloid precursor protein is located on chromosome 21, and accordingly people with Down syndrome have a very high incidence of Alzheimer's disease.

Amyloid beta is commonly thought to be intrinsically unstructured, meaning that in solution it does not acquire a unique tertiary fold but rather populates a set of structures. As such, it cannot be crystallized and most structural knowledge on amyloid beta comes from NMR and molecular dynamics. Early NMR-derived models of a 26-aminoacid polypeptide from amyloid beta (Aβ 10-35) show a collapsed coil structure devoid of significant secondary structure content. However, the most recent (2012) NMR structure of (Aβ 1-40) has significant secondary and tertiary structure. Replica exchange molecular dynamics studies suggested that amyloid beta can indeed populate multiple discrete structural states; more recent studies identified a multiplicity of discrete conformational clusters by statistical analysis. By NMR-guided simulations, amyloid beta 1-40 and amyloid beta 1-42 also seem to feature highly different conformational states, with the C-terminus of amyloid beta 1-42 being more structured than that of the 1-40 fragment.

Low-temperature and low-salt conditions allowed to isolate pentameric disc-shaped oligomers devoid of beta structure. In contrast, soluble oligomers prepared in the presence of detergents seem to feature substantial beta sheet content with mixed parallel and antiparallel character, different from fibrils; computational studies suggest an antiparallel beta-turn-beta motif instead for membrane-embedded oligomers.

The suggested mechanisms by which amyloid beta may damage and cause neuronal death include the generation of reactive oxygen species during the process of its self-aggregation. When this occurs on the membrane of neurons in vitro, it causes lipid peroxidation and the generation of a toxic aldehyde called 4-hydroxynonenal which, in turn, impairs the function of ion-motive ATPases, glucose transporters and glutamate transporters. As a result, amyloid beta promotes depolarization of the synaptic membrane, excessive calcium influx and mitochondrial impairment. Aggregations of the amyloid-beta peptide disrupt membranes in vitro.

B. Cofilin

ADF/cofilin is a family of actin-binding proteins associated with the rapid depolymerization of actin microfilaments that give actin its characteristic dynamic instability. This dynamic instability is central to actin's role in muscle contraction, cell motility and transcription regulation. As mentioned above, the presence of Cofilin in the heart and its associated with prefibrillar myocardial aggregates was reported in 2015.

Three highly conserved and highly (70%-82%) identical genes belonging to this family have been described in humans and mice:

CFL1, coding for cofilin 1 (non-muscle, or n-cofilin)

CFL2, coding for cofilin 2 (found in muscle: m-cofilin)

DSTN, coding for destrin, also known as ADF or actin depolymerizing factor Actin-binding proteins regulate assembly and disassembly of actin filaments. Cofilin, a member of the ADF/cofilin family is actually a protein with 70% sequence identity to destrin, making it part of the ADF/cofilin family of small ADP-binding proteins The protein binds to actin monomers and filaments, G actin and F actin, respectively. Cofilin causes depolymerization at the minus end of filaments, thereby preventing their reassembly. The protein is known to sever actin filaments by creating more positive ends on filament fragments. Cofilin/ADF (destrin) is likely to sever F-actin without capping and prefers ADP-actin. These monomers can be recycled by profilin, activating monomers to go back into filament form again by an ADP-to-ATP exchange. ATP-actin is then available for assembly.

The structure of actin depolymerizing factors is highly conserved across many organism due to actin's importance in many cellular processes. Proteins of the actin depolymerizing factor family characteristically consist of five beta sheets, four antiparallel and one parallel, and four alpha helices with a central alpha helix providing the structure and stability of the proteins. The actin depolymerizing factor homology domain (ADF-H domain) allows for binding to actin subunits and includes the central alpha helix, the N terminus extension, and the C terminus helix.

The N-terminus extension consists of a tilted loop that facilitates binding to G-actin but not F-actin due to steric hindrance present in F-actin. The C-terminus can form hydrogen bonds to F actin through its amide backbone and a serine at position S274. This serine is especially highly evolutionarily conserved due to its importance in actin binding. The central alpha helix is inserted into the hydrophobic cleft in between the first and third subunits of actin during actin binding.

Cofilin binds monomeric (G-actin) and filamentous actin (F-actin). Its binding affinities are higher for ADP-actin over ADP-Pi and ATP-actin. Its binding changes the twist of F-actin. The structure of ADF was first characterized in 1980 by James Bamburg. Four actin histidines near the cofilin binding site may be needed for cofilin/actin interaction, but pH sensitivity alone may not be enough of an explanation for the levels of interaction encountered. Cofilin is accommodated in ADP-F actin because of increased flexibility in this form of actin. Binding by both cofilin and ADF (destrin) causes the crossover length of the filament to be reduced. Therefore, strains increase filament dynamics and the level of filament fragmentation observed.

Cofilin is a ubiquitous actin-binding factor required for the reorganization of actin filaments. ADF/Cofilin family members bind G-actin monomers and depolymerize actin filaments through two mechanisms: severing and increasing the off-rate for actin monomers from the pointed end. “Older” ADP/ADP-Pi actin filaments free of tropomyosin and proper pH are required for cofilin to function effectively. In the presence of readily available ATP-G-actin cofilin speeds up actin polymerization via its actin-severing activity (providing free barbed ends for further polymerization and nucleation by the Arp2/3 complex). As a long-lasting in vivo effect, cofilin recycles older ADP-F-actin, helping cell to maintain ATP-G-actin pool for sustained motility. pH, phosphorylation and phosphoinositides regulate cofilin's binding and associating activity with actin.

The Arp2/3 complex and cofilin work together to reorganize the actin filaments in the cytoskeleton. Arp 2/3, an actin binding proteins complex, binds to the side of ATP-F-actin near the growing barbed end of the filament, causing nucleation of a new F-actin branch, while cofilin-driven depolymerization takes place after dissociating from the Arp2/3 complex. They also work together to reorganize actin filaments in order to traffic more proteins by vesicle to continue the growth of filaments. Cofilin also binds with other proteins such as myosin, tropomyosin, α-actinin, gelsolin and scruin. These proteins compete with cofilin for actin binding. Cofilin also play role in innate immune response.

F-actin (filamentous actin) is stabilized when it is bound to ATP due to the presence of a serine on the second subunit of actin that is able to form hydrogen bonds to the last phosphate group in ATP and a nearby histidine attached to the main loop. This interaction stabilizes the structure internally due to the interactions between the main loop and the second subunit. When ATP is hydrolyzed to ADP, the serine can no longer form a hydrogen bond to ADP due to the loss of the inorganic phosphate which causes the serine side chain to twist, causing a conformational change in the second subunit. This conformational change also causes the serine to no longer be able to form a hydrogen bond with the histidine attached to the main loop and this weakens the linkage between subunits one and three, causing the entire molecule to twist. This twisting puts strain on the molecule and destabilizes it.

Actin depolymerizing factor is able to bind to the destabilized F-actin by inserting the central helix into the cleft between the first and third subunits of actin. Actin depolymerizing factor binds F-actin cooperatively and induces a conformational change in F-actin that causes it to twist further and become more destabilized. This twisting causes severing of the bond between actin monomers, depolymerizing the filament.

C. Tau

The Tau proteins (or r proteins, after the Greek letter with that name) are a group of six highly soluble protein isoforms produced by alternative splicing from the gene MAPT (microtubule-associated protein Tau). They have roles primarily in maintaining the stability of microtubules in axons and are abundant in the neurons of the central nervous system (CNS). They are less common elsewhere but are also expressed at very low levels in CNS astrocytes and oligodendrocytes.

Pathologies and dementias of the nervous system such as Alzheimer's disease and Parkinson's disease are associated with Tau proteins that have become hyperphosphorylated insoluble aggregates called neurofibrillary tangles. The Tau proteins were identified in 1975 as heat-stable proteins essential for microtubule assembly, and since then they have been characterized as intrinsically disordered proteins. To the inventors' knowledge, Tau has not been reported as linked to cardiac disease.

Tau proteins are found more often in neurons than in non-neuronal cells in humans. One of Tau's main functions is to modulate the stability of axonal microtubules. Other nervous system microtubule-associated proteins (MAPs) may perform similar functions, as suggested by Tau knockout mice that did not show abnormalities in brain development—possibly because of compensation in Tau deficiency by other MAPs.

Although Tau is present in dendrites at low levels, where it is involved in postsynaptic scaffolding, it is active primarily in the distal portions of axons, where it provides microtubule stabilization but also flexibility as needed. Tau proteins interact with tubulin to stabilize microtubules and promote tubulin assembly into microtubules. Tau has two ways of controlling microtubule stability: isoforms and phosphorylation.

In addition to its microtubule-stabilizing function, Tau has also been found to recruit signaling proteins and to regulate microtubule-mediated axonal transport.

Tau is a negative regulator of protein translation in both Drosophila and human brains, through its binding to ribosomes, which results in impaired ribosomal function, reduction of protein synthesis and altered synaptic function. Tau interacts specifically with several ribosomal proteins, including the crucial regulator of translation rpS6.

The primary non-cellular function of Tau is to negatively regulate long-term memory and to facilitate habituation (a form of non-associative learning), two higher and more integrated physiological functions. Since regulation of Tau is critical for memory, this could explain the linkage between tauopathies and cognitive impairment.

In mice, while the reported Tau knockout strains present without overt phenotype when young, when aged, they show some muscle weakness, hyperactivity and impaired fear conditioning. However, neither spatial learning in mice, nor short-term memory (learning) in Drosophila seems to be affected by the absence of Tau.

Other typical functions of Tau include cellular signalling, neuronal development, neuroprotection and apoptosis. Atypical, non-standard roles of Tau are also under current investigation, such as its involvement in chromosome stability, its interaction with the cellular transcriptome, its interaction with other cytoskeletal or synaptic proteins, its involvement in myelination or in brain insulin signaling, its role in the exposure to chronic stress and in depression, etc.

In humans, the MAPT gene for encoding Tau protein is located on chromosome 17q21, containing 16 exons. The major Tau protein in the human brain is encoded by 11 exons. Exons 2, 3 and 10 are alternatively spliced that lead to formation of six Tau isoforms. In human brain, Tau proteins constitute a family of six isoforms with a range of 352-441 amino acids. Tau isoforms are different in either zero, one, or two inserts of 29 amino acids at the N-terminal part (exon 2 and 3) and three or four repeat-regions at the C-terminal part (exon 10). Thus, the longest isoform in the CNS has four repeats (R1, R2, R3 and R4) and two inserts (441 amino acids total), while the shortest isoform has three repeats (R1, R3 and R4) and no insert (352 amino acids total).

The MAPT gene has two haplogroups, H1 and H2, in which the gene appears in inverted orientations. Haplogroup H2 is common only in Europe and in people with European ancestry. Haplogroup H1 appears to be associated with increased probability of certain dementias, such as Alzheimer's disease. The presence of both haplogroups in Europe means that recombination between inverted haplotypes can result in the lack of one of the functioning copies of the gene, resulting in congenital defects.

Six Tau isoforms exist in human brain tissue, and they are distinguished by their number of binding domains. Three isoforms have three binding domains and the other three have four binding domains. The binding domains are located in the carboxy-terminus of the protein and are positively charged (allowing it to bind to the negatively charged microtubule). The isoforms with four binding domains are better at stabilizing microtubules than those with three binding domains. Tau is a phosphoprotein with 79 potential Serine (Ser) and Threonine (Thr) phosphorylation sites on the longest Tau isoform. Phosphorylation has been reported on approximately 30 of these sites in normal Tau proteins.

Phosphorylation of tau is regulated by a host of kinases, including PKN, a serine/threonine kinase. When PKN is activated, it phosphorylates Tau, resulting in disruption of microtubule organization. Phosphorylation of Tau is also developmentally regulated. For example, fetal Tau is more highly phosphorylated in the embryonic CNS than adult Tau. The degree of phosphorylation in all six isoforms decreases with age due to the activation of phosphatases. Like kinases, phosphatases too play a role in regulating the phosphorylation of Tau. For example, PP2A and PP2B are both present in human brain tissue and have the ability to dephosphorylate Ser396. The binding of these phosphatases to tau affects Tau's association with microtubules. Phosphorylation of Tau has also been suggested to be regulated by 0-GlcNAc modification at various Ser and Thr residues.

The accumulation of hyperphosphorylated Tau in neurons is associated with neurofibrillary degeneration. The actual mechanism of how Tau propagates from one cell to another is not well identified. Also, other mechanisms, including tau release and toxicity, are unclear. As Tau aggregates, it replaces tubulin, which in turn enhances fibrilization of tau. Several propagation methods have been proposed that occur by synaptic contact such as synaptic cell adhesion proteins, neuronal activity and other synaptic and non-synaptic mechanisms. The mechanism of Tau aggregation is still not completely elucidated, but several factors favor this process, including tau phosphorylation and zinc ions.

Tau causes toxic effects through its accumulation inside cells. Many enzymes are involved in toxicity mechanism such as PAR-1 kinase. This enzyme stimulates phosphorylation of serine 262 and 356, which in turn leads to activate other kinases (GSK-3 and CDK5) that cause disease-associated phosphoepitopes. The degree of toxicity is affected by different factors, such as the degree of microtubule binding. Toxicity could also happen by neurofibrillary tangles (NFTs), which leads to cell death and cognitive decline.

Hyperphosphorylation of the Tau protein (Tau inclusions, pTau) can result in the self-assembly of tangles of paired helical filaments and straight filaments, which are involved in the pathogenesis of Alzheimer's disease, frontotemporal dementia and other tauopathies. All of the six Tau isoforms are present in an often hyperphosphorylated state in paired helical filaments in the Alzheimer's disease brain. In other neurodegenerative diseases, the deposition of aggregates enriched in certain Tau isoforms has been reported. When misfolded, this otherwise very soluble protein can form extremely insoluble aggregates that contribute to a number of neurodegenerative diseases. Tau protein has a direct effect on the breakdown of a living cell caused by tangles that form and block nerve synapses.

Gender-specific Tau gene expression across different regions of the human brain has recently been implicated in gender differences in the manifestations and risk for tauopathies. Some aspects of how the disease functions also suggest that it has some similarities to prion proteins.

The tau hypothesis states that excessive or abnormal phosphorylation of Tau results in the transformation of normal adult Tau into paired-helical-filament (PHF) Tau and neurofibrillary tangles (NFTs). The stage of the disease determines NFTs' phosphorylation. In AD, at least 19 amino acids are phosphorylated; pre-NFT phosphorylation occurs at serine 119, 202 and 409, while intra-NFT phosphorylation happens at serine 396 and threonine 231. Through its isoforms and phosphorylation, Tau protein interacts with tubulin to stabilize microtubule assembly. All of the six Tau isoforms are present in an often hyperphosphorylated state in paired helical filaments (PHFs) in the AD brain.

Tau mutations have many consequences, including microtubule dysfunction and alteration of the expression level of Tau isoforms. Mutations that alter function and isoform expression of Tau lead to hyperphosphorylation. The process of Tau aggregation in the absence of mutations is not known but might result from increased phosphorylation, protease action or exposure to polyanions, such as glycosaminoglycans. Hyperphosphorylated Tau disassembles microtubules and sequesters normal Tau, MAPT 1 (microtubule associated protein tau 1), MAPT 2 and ubiquitin into tangles of PHFs. This insoluble structure damages cytoplasmic functions and interferes with axonal transport, which can lead to cell death.

Hyperphosphorylated forms of Tau protein are the main component of PHIFs of NFTs in the brain of AD patients. It has been well demonstrated that regions of Tau six-residue segments, namely PIHF6 (VQIVYK) and PHF6* (VQIINK), can form Tau PHF aggregation in AD. Apart from the PHF6, some other residue sites like Ser285, Ser289, Ser293, Ser305 and Tyr310, located near the C-terminal of the PHIF6 sequences, play key roles in the phosphorylation of tau. Hyperphosphorylated Tau differs in its sensitivity and its kinase as well as alkaline phosphatase activity and is, along with beta-amyloid, a component of the pathologic lesion seen in Alzheimer disease. A68 is a name sometimes given (mostly in older publications) to the hyperphosphorylated form of Tau protein found in the brains of individuals with Alzheimer's disease.

### II. MONOCLONAL ANTIBODIES AND PRODUCTION THEREOF

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the α and γ chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in C_{H} sequence and function, humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the VsubH when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

A. General Methods

It will be understood that monoclonal antibodies will have several applications. These include the production of diagnostic kits for use in detecting and diagnosing heart disease, as well as for treating the same. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce antibody secreting B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

In the case of human antibodies against natural pathogens, a suitable approach is to identify subjects that have been exposed to the pathogens, such as those who have been diagnosed as having contracted the disease, or those who have been vaccinated to generate protective immunity against the pathogen or to test the safety or efficacy of an experimental vaccine. Circulating anti-pathogen antibodies can be detected, and antibody encoding or producing B cells from the antibody-positive subject may then be obtained.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens, lymph nodes, tonsils or adenoids, bone marrow aspirates or biopsies, tissue biopsies from mucosal organs like lung or GI tract, or from circulating blood. The antibody-producing B lymphocytes from the immunized animal or immune human are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984). HMMA2.5 cells or MFP-2 cells are particularly useful examples of such cells.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986) and there are processes for better efficiency (Yu et al., 2008). Fusion procedures usually produce viable hybrids at low frequencies, about 1×10⁻⁶ to 1×10⁻⁸, but with optimized procedures one can achieve fusion efficiencies close to 1 in 200 (Yu et al., 2008). However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

B. Antibodies to Cofilin, Tau and Aβ

Antibodies according to the present disclosure may be defined, in the first instance, by their binding specificity to Cofilin, Tau and Aβ. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

There are commercially available antibodies to Cofilin (MA5-17275 and GT217 from Thermo Fisher Scientific, ab54532 from Abcam, E-8 from Santa Cruz Biotechnology, 77G2 from Cell Signal), Tau (TOMA) and Aβ (VIA/ABN1650 from EMID Millipore), among others. Antibody A11 detects oligomeric Aβ40/42 but not monomers. In particular, the activity TOMA has been reported in several papers including Castillo-Carranza et al, “Passive immunization with Tau oligomer monoclonal antibody reverses tauopathy phenotypes without affecting hyperphosphorylated neuroibrillary tangles,” J. Neurosci. 34(12):4260-72 (2014), and Schroeder et al., “Oligomeric tau-targeted immunotherapy in Tg4510 mice,” Alzheimer's Research & Therapy 9:46 (2017).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if an antibody competes for binding with a reference antibody, the above-described binding methodology is performed in two orientations: In a first orientation, the reference antibody is allowed to bind to the antigen under saturating conditions followed by assessment of binding of the test antibody to the antigen. In a second orientation, the test antibody is allowed to bind to the antigen molecule under saturating conditions followed by assessment of binding of the reference antibody to the antigen. If, in both orientations, only the first (saturating) antibody is capable of binding to the antigen, then it is concluded that the test antibody and the reference antibody compete for binding to the antigen. As will be appreciated by a person of ordinary skill in the art, an antibody that competes for binding with a reference antibody may not necessarily bind to the identical epitope as the reference antibody but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

In another aspect, the antibodies may be defined by their variable sequence, which include additional “framework” regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below).

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example, with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the below-described antibodies and their antigen-binding fragments. The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example, antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002), J. Biol. Chem. 277(30): 26733-26740; Davies J. et al. (2001), Biotechnology & Bioengineering 74(4): 288-294). Methods of altering carbohydrate contents are known to those skilled in the art, see, e.g., Wallick, S. C. et al. (1988), J. Exp. Med. 168(3): 1099-1109; Tao, M. H. et al. (1989), J. Immunol. 143(8): 2595-2601; Routledge, E. G. et al. (1995), Transplantation 60(8):847-53; Elliott, S. et al. (2003), Nature Biotechnol. 21:414-21; Shields, R. L. et al. (2002), J. Biol. Chem. 277(30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

C. Engineering of Antibody Sequences

In various embodiments, one may choose to engineer sequences of the identified antibodies for a variety of reasons, such as improved expression, improved cross-reactivity or diminished off-target binding. Modified antibodies may be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated NimΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. F(ab′) antibody derivatives are monovalent, while F(ab′)₂ antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23p19 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

FcRn binding. Fc mutations can also be introduced and engineered to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, (J. Biol. Chem. 276:6591-6604). A number of methods are known that can result in increased half-life (Kuo and Aveson, (2011)), including amino acid modifications may be generated through techniques including alanine scanning mutagenesis, random mutagenesis and screening to assess the binding to the neonatal Fc receptor (FcRn) and/or the in vivo behavior. Computational strategies followed by mutagenesis may also be used to select one of amino acid mutations to mutate.

The present disclosure therefore provides a variant of an antigen binding protein with optimized binding to FcRn. In a particular embodiment, the said variant of an antigen binding protein comprises at least one amino acid modification in the Fc region of said antigen binding protein, wherein said modification is selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401403, 404, 408, 411, 412, 414, 415, 416, 418, 419, 420, 421, 422, 424, 426, 428, 433, 434, 438, 439, 440, 443, 444, 445, 446 and 447 of the Fc region as compared to said parent polypeptide, wherein the numbering of the amino acids in the Fc region is that of the EU index in Kabat. In a further aspect of the disclosure the modifications are M252Y/S254T/T256E.

Additionally, various publications describe methods for obtaining physiologically active molecules whose half-lives are modified, see for example Kontermann (2009) either by introducing an FcRn-binding polypeptide into the molecules or by fusing the molecules with antibodies whose FcRn-binding affinities are preserved but affinities for other Fc receptors have been greatly reduced or fusing with FcRn binding domains of antibodies.

Derivatized antibodies may be used to alter the half-lives (e.g., serum half-lives) of parental antibodies in a mammal, particularly a human. Such alterations may result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present disclosure or fragments thereof in a mammal, preferably a human, results in a higher serum titer of said antibodies or antibody fragments in the mammal, and thus reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor.

Beltramello et al. (2010) previously reported the modification of neutralizing mAbs, due to their tendency to enhance dengue virus infection, by generating in which leucine residues at positions 1.3 and 1.2 of CH2 domain (according to the IMGT unique numbering for C-domain) were substituted with alanine residues. This modification, also known as “LALA” mutation, abolishes antibody binding to FcγRI, FcγRII and FcγRIIIa, as described by Hessell et al. (2007). The variant and unmodified recombinant mAbs were compared for their capacity to neutralize and enhance infection by the four dengue virus serotypes. LALA variants retained the same neutralizing activity as unmodified mAb but were completely devoid of enhancing activity. LALA mutations of this nature are therefore contemplated in the context of the presently disclosed antibodies.

Altered Glycosylation. A particular embodiment of the present disclosure is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan may be covalently attached to the heavy chain constant region.

Another embodiment of the present disclosure comprises a mAb with a novel Fc glycosylation pattern. The isolated monoclonal antibody, or antigen binding fragment thereof, is present in a substantially homogenous composition represented by the GNGN or G1/G2 glycoform. Fc glycosylation plays a significant role in anti-viral and anti-cancer properties of therapeutic mAbs. The disclosure is in line with a recent study that shows increased anti-lentivirus cell-mediated viral inhibition of a fucose free anti-HIV mAb in vitro. This embodiment of the present disclosure with homogenous glycans lacking a core fucose, showed increased protection against specific viruses by a factor greater than two-fold. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for Fc γ RI and Fc γ RIII compared to the same antibody without the substantially homogeneous GNGN glycoform and with G0, G1F, G2F, GNF, GNGNF or GNGNFX containing glycoforms. In one embodiment of the present disclosure, the antibody dissociates from Fc γ RI with a Kd of 1×10⁻⁸ M or less and from Fc γ RIII with a Kd of 1×10⁻⁷ M or less.

Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the antibody is expressed in cells that express beta (1,4)-N-acetylglucosaminyltransferase III (GnT III), such that GnT III adds GlcNAc to the IL-23p19 antibody. Methods for producing antibodies in such a fashion are provided in WO/9954342, WO/03011878, U.S. Patent Publication No. US20030003097A1, and Umana et al., Nature Biotechnology, 17:176-180, February 1999. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express recombinant monoclonal antibodies.

Elimination of monoclonal antibody protein sequence liabilities. It is possible to engineer the antibody variable gene sequences obtained from human B cells to enhance their manufacturability and safety. Potential protein sequence liabilities can be identified by searching for sequence motifs associated with sites containing:
1) Unpaired Cys residues,
2) N-linked glycosylation,
3) Asn deamidation,
4) Asp isomerization,
5) SYE truncation,
6) Met oxidation,
7) Trp oxidation,
8) N-terminal glutamate,
9) Integrin binding,
10) CD11c/CD18 binding, or
11) Fragmentation
   Such motifs can be eliminated by altering the synthetic gene for the cDNA encoding recombinant antibodies.

Protein engineering efforts in the field of development of therapeutic antibodies clearly reveal that certain sequences or residues are associated with solubility differences (Fernandez-Escamilla et al., Nature Biotech., 22 (10), 1302-1306, 2004; Chennamsetty et al., PNAS, 106 (29), 11937-11942, 2009; Voynov et al., Biocon. Chem., 21 (2), 385-392, 2010) Evidence from solubility-altering mutations in the literature indicate that some hydrophilic residues such as aspartic acid, glutamic acid, and serine contribute significantly more favorably to protein solubility than other hydrophilic residues, such as asparagine, glutamine, threonine, lysine, and arginine.

Stability. Antibodies can be engineered for enhanced biophysical properties. One can use elevated temperature to unfold antibodies to determine relative stability, using average apparent melting temperatures. Differential Scanning Calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

Solubility. One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Autoreactivity. Generally, it is thought that autoreactive clones should be eliminated during ontogeny by negative selection, however it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

Preferred residues (“Human Likeness”). B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

D. Single Chain Antibodies

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

E. Multispecific Antibodies

In certain embodiments, antibodies of the present disclosure are bispecific or multispecific. Bispecific antibodies are antibodies that have binding specificities for at least two different epitopes. Exemplary bispecific antibodies may bind to two different epitopes of a single antigen. Other such antibodies may combine a first antigen binding site with a binding site for a second antigen. Alternatively, an anti-pathogen arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc 7 RIII (CD16), so as to focus and localize cellular defense mechanisms to the infected cell. Bispecific antibodies may also be used to localize cytotoxic agents to infected cells. These antibodies possess a pathogen-binding arm and an arm that binds the cytotoxic agent (e.g., saporin, anti-interferon-α, vinca alkaloid, ricin A chain, methotrexate or radioactive isotope hapten). Bispecific antibodies can be prepared as full-length antibodies or antibody fragments (e.g., F(ab′)₂ bispecific antibodies). WO 96/16673 describes a bispecific anti-ErbB2/anti-Fc γ RIII antibody and U.S. Pat. No. 5,837,234 discloses a bispecific anti-ErbB2/anti-Fc γ RI antibody. A bispecific anti-ErbB2/Fc alpha antibody is shown in WO98/02463. U.S. Pat. No. 5,821,337 teaches a bispecific anti-ErbB2/anti-CD3 antibody.

Methods for making bispecific antibodies are known in the art. Traditional production of full-length bispecific antibodies is based on the co-expression of two immunoglobulin heavy chain-light chain pairs, where the two chains have different specificities (Millstein et al., Nature, 305:537-539, 1983). Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

In a particular embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175: 217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998). doi:10.1038/nbt0798-677pmid:9661204). For example, bispecific antibodies have been produced using leucine zippers (Kostelny et al., J. Immunol., 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

In a particular embodiment, a bispecific or multispecific antibody may be formed as a DOCK-AND-LOCK™ (DNL™) complex (see, e.g., U.S. Pat. Nos. 7,521,056; 7,527,787; 7,534,866; 7,550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264; Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147: 60, 1991; Xu et al., Science, 358(6359):85-90, 2017). A multivalent antibody may be internalized (and/or catabolized) faster than a bivalent antibody by a cell expressing an antigen to which the antibodies bind. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1)ₙ-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

Accordingly, in particular embodiments, an antibody comprised in the therapeutic agent comprises
(a) a first Fab molecule which specifically binds to a first antigen;
(b) a second Fab molecule which specifically binds to a second antigen, and wherein the variable domains VL and VH of the Fab light chain and the Fab heavy chain are replaced by each other,
wherein the first antigen is an activating T cell antigen and the second antigen is a target cell antigen, or the first antigen is a target cell antigen and the second antigen is an activating T cell antigen; and
wherein
   i) in the constant domain CL of the first Fab molecule under a) the amino acid at position 124 is substituted by a positively charged amino acid (numbering according to Kabat), and wherein in the constant domain CH1 of the first Fab molecule under a) the amino acid at position 147 or the amino acid at position 213 is substituted by a negatively charged amino acid (numbering according to Kabat EU index); or
   ii) in the constant domain CL of the second Fab molecule under b) the amino acid at position 124 is substituted by a positively charged amino acid (numbering according to Kabat), and wherein in the constant domain CH1 of the second Fab molecule under b) the amino acid at position 147 or the amino acid at position 213 is substituted by a negatively charged amino acid (numbering according to Kabat EU index).
      The antibody may not comprise both modifications mentioned under i) and ii). The constant domains CL and CH1 of the second Fab molecule are not replaced by each other (i.e., remain unexchanged).

In another embodiment of the antibody, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 or the amino acid at position 213 is substituted independently by glutamic acid (E), or aspartic acid (D) (numbering according to Kabat EU index).

In a further embodiment, in the constant domain CL of the first Fab molecule under a) the amino acid at position 124 is substituted independently by lysine (K), arginine (R) or histidine (H) (numbering according to Kabat), and in the constant domain CH1 of the first Fab molecule under a) the amino acid at position 147 is substituted independently by glutamic acid (E), or aspartic acid (D) (numbering according to Kabat EU index).

In a particular embodiment, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)) and the amino acid at position 123 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In a more particular embodiment, in the constant domain CL of the first Fab molecule under a) the amino acid at position 124 is substituted by lysine (K) (numbering according to Kabat) and the amino acid at position 123 is substituted by lysine (K) or arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

In an even more particular embodiment, in the constant domain CL of the first Fab molecule under a) the amino acid at position 124 is substituted by lysine (K) (numbering according to Kabat) and the amino acid at position 123 is substituted by arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

F. Chimeric Antigen Receptors

Artificial T cell receptors (also known as chimeric T cell receptors, chimeric immunoreceptors, chimeric antigen receptors (CARs)) are engineered receptors, which graft an arbitrary specificity onto an immune effector cell. Typically, these receptors are used to graft the specificity of a monoclonal antibody onto a T cell, with transfer of their coding sequence facilitated by retroviral vectors. In this way, a large number of target-specific T cells can be generated for adoptive cell transfer. Phase I clinical studies of this approach show efficacy.

The most common form of these molecules are fusions of single-chain variable fragments (scFv) derived from monoclonal antibodies, fused to CD3-zeta transmembrane and endodomain. Such molecules result in the transmission of a zeta signal in response to recognition by the scFv of its target. An example of such a construct is 14g2a-Zeta, which is a fusion of a scFv derived from hybridoma 14g2a (which recognizes disialoganglioside GD2). When T cells express this molecule (usually achieved by oncoretroviral vector transduction), they recognize and kill target cells that express GD2 (e.g., neuroblastoma cells). To target malignant B cells, investigators have redirected the specificity of T cells using a chimeric immunoreceptor specific for the B-lineage molecule, CD19.

The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows to the scFv to orient in different directions to enable antigen binding. The transmembrane domain is a typical hydrophobic alpha helix usually derived from the original molecule of the signaling endodomain which protrudes into the cell and transmits the desired signal.

Type I proteins are in fact two protein domains linked by a transmembrane alpha helix in between. The cell membrane lipid bilayer, through which the transmembrane domain passes, acts to isolate the inside portion (endodomain) from the external portion (ectodomain). It is not so surprising that attaching an ectodomain from one protein to an endodomain of another protein results in a molecule that combines the recognition of the former to the signal of the latter.

Ectodomain. A signal peptide directs the nascent protein into the endoplasmic reticulum. This is essential if the receptor is to be glycosylated and anchored in the cell membrane. Any eukaryotic signal peptide sequence usually works fine. Generally, the signal peptide natively attached to the amino-terminal most component is used (e.g., in a scFv with orientation light chain-linker-heavy chain, the native signal of the light-chain is used

The antigen recognition domain is usually an scFv. There are however many alternatives. An antigen recognition domain from native T-cell receptor (TCR) alpha and beta single chains have been described, as have simple ectodomains (e.g., CD4 ectodomain to recognize MV infected cells) and more exotic recognition components such as a linked cytokine (which leads to recognition of cells bearing the cytokine receptor). In fact, almost anything that binds a given target with high affinity can be used as an antigen recognition region.

A spacer region links the antigen binding domain to the transmembrane domain. It should be flexible enough to allow the antigen binding domain to orient in different directions to facilitate antigen recognition. The simplest form is the hinge region from IgG1. Alternatives include the CH₂CH₃ region of immunoglobulin and portions of CD3. For most scFv based constructs, the IgG1 hinge suffices. However, the best spacer often has to be determined empirically.

Transmembrane domain. The transmembrane domain is a hydrophobic alpha helix that spans the membrane. Generally, the transmembrane domain from the most membrane proximal component of the endodomain is used. Interestingly, using the CD3-zeta transmembrane domain may result in incorporation of the artificial TCR into the native TCR a factor that is dependent on the presence of the native CD3-zeta transmembrane charged aspartic acid residue. Different transmembrane domains result in different receptor stability. The CD28 transmembrane domain results in a brightly expressed, stable receptor.

Endodomain. This is the “business-end” of the receptor. After antigen recognition, receptors cluster and a signal is transmitted to the cell. The most commonly used endodomain component is CD3-zeta which contains 3 ITAMs. This transmits an activation signal to the T cell after antigen is bound. CD3-zeta may not provide a fully competent activation signal and additional co-stimulatory signaling is needed.

“First-generation” CARs typically had the intracellular domain from the CD3 ξ-chain, which is the primary transmitter of signals from endogenous TCRs. “Second-generation” CARs add intracellular signaling domains from various costimulatory protein receptors (e.g., CD28, 41BB, ICOS) to the cytoplasmic tail of the CAR to provide additional signals to the T cell. Preclinical studies have indicated that the second generation of CAR designs improves the antitumor activity of T cells. More recent, “third-generation” CARs combine multiple signaling domains, such as CD3z-CD28-41BB or CD3z-CD28-OX40, to further augment potency.

G. ADCs

Antibody Drug Conjugates or ADCs are a new class of highly potent biopharmaceutical drugs designed as a targeted therapy for the treatment of people with infectious disease. ADCs are complex molecules composed of an antibody (a whole mAb or an antibody fragment such as a single-chain variable fragment, or scFv) linked, via a stable chemical linker with labile bonds, to a biological active cytotoxic/anti-viral payload or drug. Antibody Drug Conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the infected cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on infected cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cell or impairs viral replication. Due to this targeting, ideally the drug has lower side effects and gives a wider therapeutic window than other agents.

A stable link between the antibody and cytotoxic/anti-viral agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and noncleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker.

The availability of better and more stable linkers has changed the function of the chemical bond. The type of linker, cleavable or noncleavable, lends specific properties to the cytotoxic (anti-cancer) drug. For example, a non-cleavable linker keeps the drug within the cell. As a result, the entire antibody, linker and cytotoxic agent enter the targeted cancer cell where the antibody is degraded to the level of an amino acid. The resulting complex-amino acid, linker and cytotoxic agent—now becomes the active drug. In contrast, cleavable linkers are catalyzed by enzymes in the host cell where it releases the cytotoxic agent.

Another type of cleavable linker, currently in development, adds an extra molecule between the cytotoxic/anti-viral drug and the cleavage site. This linker technology allows researchers to create ADCs with more flexibility without worrying about changing cleavage kinetics. Researchers are also developing a new method of peptide cleavage based on Edman degradation, a method of sequencing amino acids in a peptide. Future direction in the development of ADCs also include the development of site-specific conjugation (TDCs) to further improve stability and therapeutic index and a emitting immunoconjugates and antibody-conjugated nanoparticles.

H. BiTES

Bi-specific T-cell engagers (BiTEs) are a class of artificial bispecific monoclonal antibodies that are investigated for the use as anti-cancer drugs. They direct a host's immune system, more specifically the T cells' cytotoxic activity, against infected cells. BiTE is a registered trademark of Micromet AG.

BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to an infected cell via a specific molecule.

Like other bispecific antibodies, and unlike ordinary monoclonal antibodies, BiTEs form a link between T cells and target cells. This causes T cells to exert cytotoxic/anti-viral activity on infected cells by producing proteins like perforin and granzymes, independently of the presence of MHC I or co-stimulatory molecules. These proteins enter infected cells and initiate the cell's apoptosis. This action mimics physiological processes observed during T cell attacks against infected cells.

I. Intrabodies

In a particular embodiment, the antibody is a recombinant antibody that is suitable for action inside of a cell—such antibodies are known as “intrabodies.” These antibodies may interfere with target function by a variety of mechanism, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required.

The two major issues impacting the implementation of intrabody therapeutic are delivery, including cell/tissue targeting, and stability. With respect to delivery, a variety of approaches have been employed, such as tissue-directed delivery, use of cell-type specific promoters, viral-based delivery and use of cell-permeability/membrane translocating peptides. With respect to the stability, the approach is generally to either screen by brute force, including methods that involve phage display and may include sequence maturation or development of consensus sequences, or more directed modifications such as insertion stabilizing sequences (e.g., Fc regions, chaperone protein sequences, leucine zippers) and disulfide replacement/modification.

An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.; Persic et al., 1997).

By virtue of their ability to enter cells, intrabodies have additional uses that other types of antibodies may not achieve. In the case of the present antibodies, the ability to interact with the MUC1 cytoplasmic domain in a living cell may interfere with functions associated with the MUC1 CD, such as signaling functions (binding to other molecules) or oligomer formation. In particular, it is contemplated that such antibodies can be used to inhibit MUC1 dimer formation.

J. Purification

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### III. TREATMENT/PREVENTION OF HEART DISEASE

A. Formulation and Administration

The present disclosure provides pharmaceutical compositions comprising antibodies. Such compositions comprise a prophylactically or therapeutically effective amount of an antibody or a fragment thereof, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In one embodiment, the methods for the treatment of heart disease comprise reducing one or more of the symptoms of heart disease, such as reduced exercise capacity, reduced blood ejection volume, increased left ventricular end diastolic pressure, increased pulmonary capillary wedge pressure, reduced cardiac output, cardiac index, increased pulmonary artery pressures, increased left ventricular end systolic and diastolic dimensions, and increased left ventricular wall stress, wall tension and wall thickness-same for right ventricle. Treatment regimens would vary depending on the clinical situation. However, long term maintenance would appear to be appropriate in most circumstances. It also may be desirable treat intermittently, such as within brief window during disease progression.

B. Combination Therapies

In another embodiment, it is envisioned to use an antibody as described herein in combination with other therapeutic modalities. Thus, in addition to the antibody therapy, one may also provide to the patient more “standard” pharmaceutical cardiac therapies. Examples of standard therapies include, without limitation, so-called “beta blockers,” anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists and cytokine blockers/inhibitors.

Combinations may be achieved by administering a single composition or pharmacological formulation that includes both agents, or by administering two distinct compositions or formulations, at the same time, wherein one composition includes the antibody and the other includes the agent. Alternatively, the antibody therapy may precede or follow administration of the other agent by intervals ranging from minutes to weeks. In embodiments where the other agent and antibody are administered separately, one would generally ensure that a significant period of time did not expire between the time of each delivery, such that the antibody and other agent would still be able to exert an advantageously combined effect on the patient. In such instances, it is contemplated that one would typically administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either an antibody or the other agent will be desired. In this regard, various combinations may be employed. By way of illustration, where the antibody is “A” and the other agent is “B”, the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated.

### IV. ANTIBODY CONJUGATES

Antibodies of the present disclosure may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### V. IMMUNODETECTION METHODS

In still further embodiments, the present disclosure concerns immunodetection methods for binding, purifying, removing, quantifying and otherwise generally detecting misfolded proteins associated with heart disease such as Aβ, Tau, and Cofilin. While such methods can be applied in a traditional sense, another use will be in quality control and monitoring of vaccine stocks, where antibodies according to the present disclosure can be used to assess the amount or integrity (i.e., long term stability) of antigens. Alternatively, the methods may be used to screen various antibodies for appropriate/desired reactivity profiles.

Other immunodetection methods include specific assays for determining the presence of misfolded proteins in a subject. A wide variety of assay formats are contemplated, but specifically those that would be used to detect misfolded proteins in a sample obtained from a subject. The assays may be advantageously formatted for non-healthcare (home) use, including lateral flow assays (see below) analogous to home pregnancy tests. These assays may be packaged in the form of a kit with appropriate reagents and instructions to permit use by the subject of a family member.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of antibodies directed to specific epitopes in samples also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing misfolded proteins, and contacting the sample with a first antibody in accordance with the present disclosure, as the case may be, under conditions effective to allow the formation of immunocomplexes.

These methods include methods for purifying misfolded proteins from a sample. The antibody will preferably be linked to a solid support, such as in the form of a column matrix, and the sample suspected of containing the misfolded proteins will be applied to the immobilized antibody. The unwanted components will be washed from the column, leaving the misfolded proteins immunocomplexed to the immobilized antibody, which is then collected by removing the organism or antigen from the column.

The immunobinding methods also include methods for detecting and quantifying the amount of misfolded proteins or related components in a sample and the detection and quantification of any immune complexes formed during the binding process. Here, one would obtain a sample suspected of containing misfolded proteins and contact the sample with an antibody that binds misfolded proteins or components thereof, followed by detecting and quantifying the amount of immune complexes formed under the specific conditions. In terms of antigen detection, the biological sample analyzed may be any sample that is suspected of containing misfolded proteins, such as a tissue section or specimen, a homogenized tissue extract, a biological fluid.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to misfolded proteins present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, for example, with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

A. ELISAs

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the misfolded proteins is added to the wells. After binding and washing to remove non-specifically bound immune complexes, the bound antigen may be detected. Detection may be achieved by the addition of another anti-misfolded protein antibody that is linked to a detectable label. This type of ELISA is a simple “sandwich ELISA.” Detection may also be achieved by the addition of a second anti-misfolded protein antibody, followed by the addition of a third antibody that has binding affinity for the second antibody, with the third antibody being linked to a detectable label.

In another exemplary ELISA, the samples suspected of containing the misfolded proteins are immobilized onto the well surface and then contacted with the anti-misfolded protein antibodies of the disclosure. After binding and washing to remove non-specifically bound immune complexes, the bound anti-misfolded protein antibodies are detected. Where the initial anti-misfolded protein antibodies are linked to a detectable label, the immune complexes may be detected directly. Again, the immune complexes may be detected using a second antibody that has binding affinity for the first anti-misfolded protein antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

In another embodiment, the present disclosure contemplates the use of competitive formats. In competition-based assays, an unknown amount of analyte or antibody is determined by its ability to displace a known amount of labeled antibody or analyte. Thus, the quantifiable loss of a signal is an indication of the amount of unknown antibody or analyte in a sample.

B. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

C. Lateral Flow Assays

Lateral flow assays, also known as lateral flow immunochromatographic assays, are simple devices intended to detect the presence (or absence) of a target analyte in sample (matrix) without the need for specialized and costly equipment, though many laboratory-based applications exist that are supported by reading equipment. Typically, these tests are used as low resources medical diagnostics, either for home testing, point of care testing, or laboratory use. A widely spread and well-known application is the home pregnancy test.

The technology is based on a series of capillary beds, such as pieces of porous paper or sintered polymer. Each of these elements has the capacity to transport fluid (e.g., urine) spontaneously. The first element (the sample pad) acts as a sponge and holds an excess of sample fluid. Once soaked, the fluid migrates to the second element (conjugate pad) in which the manufacturer has stored the so-called conjugate, a dried format of bio-active particles (see below) in a salt-sugar matrix that contains everything to guarantee an optimized chemical reaction between the target molecule (e.g., an antigen) and its chemical partner (e.g., antibody) that has been immobilized on the particle's surface. While the sample fluid dissolves the salt-sugar matrix, it also dissolves the particles and in one combined transport action the sample and conjugate mix while flowing through the porous structure. In this way, the analyte binds to the particles while migrating further through the third capillary bed. This material has one or more areas (often called stripes) where a third molecule has been immobilized by the manufacturer. By the time the sample-conjugate mix reaches these strips, analyte has been bound on the particle and the third ‘capture’ molecule binds the complex. After a while, when more and more fluid has passed the stripes, particles accumulate and the stripe-area changes color. Typically there are at least two stripes: one (the control) that captures any particle and thereby shows that reaction conditions and technology worked fine, the second contains a specific capture molecule and only captures those particles onto which an analyte molecule has been immobilized. After passing these reaction zones, the fluid enters the final porous material—the wick—that simply acts as a waste container. Lateral Flow Tests can operate as either competitive or sandwich assays. Lateral flow assays are disclosed in U.S. Pat. No. 6,485,982.

D. Immunohistochemistry

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

E. Immunodetection Kits

In still further embodiments, the present disclosure concerns immunodetection kits for use with the immunodetection methods described above. As the antibodies may be used to detect misfolded proteins, the antibodies may be included in the kit. The immunodetection kits will thus comprise, in suitable container means, a first antibody that binds to misfolded proteins, and optionally an immunodetection reagent.

In certain embodiments, the antibody may be pre-bound to a solid support, such as a column matrix and/or well of a microtiter plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of the misfolded proteins, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

### VI. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Methods

Human Protection. Failing hearts were obtained from explanted hearts from patients with primary diagnosis of iDCM at the Heart Center at Massachusetts General Hospital in Boston (MA). Co-morbidities present in the failing hearts were not the primary cause of myocardial dysfunction. Tissue processing was performed at Beth Israel Deaconess Medical Center and the Institutional Review Board at both hospitals approved the study. Fully anonymized hearts and brain tissue and available clinical data for controls and AD were obtained from organ donors through the National Disease Research Interchange (NDRI) supported by the National Institute of Health. All donations were with family approval. The inventors maintained strict confidentiality to protect the privacy of patients and donors. The study was conformed to the declaration of Helsinki.

Human Tissue. Myocardial samples from iDCM cases collected at time of explant were cardio-protected by cross clamp infusion of cold oxygenated dilute blood cardioplegic solution before heart harvesting and kept in cold oxygenated Wisconsin cardioplegic solution for transport to the laboratory. Donor hearts were obtained from subjects with no clinical history, macroscopic, laboratory or instrumental signs of cardiac diseases. Control hearts were found not suitable for transplantation due to lack of identification of a compatible recipient, blood transfusion while in the emergency department, age of donor, or need for resuscitation. Control hearts were also cardio-protected at time of explant and transported in cold Wisconsin Cardioplegic solution. Whole heart was obtained from four cases with clinical diagnosis of AD, but no history of cardiac disease as previously published by our group (Troncone et al., 2016). Age, gender, ethnicity matched hearts from donors with no history or clinical signs of AD or DCM were used as controls. Upon arrival to the laboratory, the hearts were dissected in the different regions (Anterior Wall—AW, Posterior Wall—PW, Lateral Wall—LW, Septal Wall—SW of the left ventricle—LV, and right ventricular wall—RV). Samples from each region were frozen in liquid nitrogen and stored at −80° C. for molecular biology tests and immunohistochemistry. A sample from the anterior wall was also collected in 4% paraformaldehyde-lysine-sodium metaperiodate in 0.1M NaPO4 (PLP) for immunohistochemistry.

Mouse model. hTau and C57Bl/6J control mice were obtained from Jackson Laboratory (005491 and 00664, respectively). hTau mice lack the mouse endogenous microtubule-associated protein tau (Mapt) gene expression, and express all six isoforms (including both 3R and 4R forms) of human MAPT (Andorfer, C., 2003; 2005). The transgene contains the coding sequence, intronic regions, and regulatory elements of the human microtubule-associated protein tau (MAPT) gene. Male and female mice were used in the study. All data presented were collected in three, seven and twelve-month old mice. Cardiomyocytes were isolated from six-month-old mice. The study complies with the National Institutes of Health Guide for the Care and Use of Laboratory Animals and was approved by the local Institutional Animal Care and Use Committee.

Echocardiography. C57Bl/6J wild-type (WT) and hTau mice underwent 2D guided M-mode transthoracic echocardiography (Visual Sonic Vevo 3100). Echocardiography was performed in light anesthetized mice (2% isofluorane). M-Mode measurements were performed from images obtained in short axis at the level of the papillary muscles. The inventors measured the parameters that define diastolic dysfunction by echocardiography including: left atria (LA) dimension and pressure; mitral valve flow deceleration time; E/A ratio; E/e′ ratio. Mitral valve flow velocity (E, A) and tissue doppler velocity (e′) were measured in apical four chamber view. Tissue doppler measurements were taken from the septum at the level of the mitral valve anulus. Male and female mice were tested for the three and twelve-month group. Of note estrogen contribution should be absent in our mice since female were housed in groups and separated from the male, therefore the estrous cycle was suppressed or inhibited (Van Der Lee, S., 1955; 1956; Whitten, 1959). Mice were not used for breeding.

Mouse Behavioral tests. All tests were conducted from 10:00 am-03:00 μm in the lights-on cycle at VA Small Animal Behavioral & Physiological Assessment Core Facility by experimenters blinded to group designation. Mice were habituated to the procedure room 30 min before each test.
Open Field Test (Polydoro, 2009: Gernson, 2018). Mice will be placed in the center of a dim lit (30-40 lux) chamber of the open-field apparatus (44×44×30 cm). Movements of the animals is tracked by an automatic monitoring system for 5 min. A computer-assisted video tracking system (Noldus Ethovision XT) recorded and measured distance traveled (cm) and time spent (s) in central and peripheral zones.
Novelty Y-maze Test. Mice were tested in Y-maze apparatus (50×11×10 cm) made of white Plexiglas, which included two trials. During training trial, mice were pseudo-randomly assigned the two arms, allowing for a 2 minutes exploration of only two arms of the maze. After a 5-minutes delay, test trial was started. During test trial, the mice allowed to explore freely all three arms of the maze for 2 minutes. A computer-assisted video tracking system (Noldus Ethovision XT) recorded and measured distance traveled (cm) and time spent (sn) in novel arm (previously blocked arm).
Morris Water Maze Test. The circular white pool (80 cm diameter, 50 cm deep) with murky water filled to 20 cm depth, at ˜24° C. Intra-maze and extra-maze cues were included. The target (a round, clear, plastic platform 8 cm in diameter) was placed 1.5 cm below the surface of the water. Mice were placed in the tank facing the wall and allocated 80 seconds to find the platform, mount the platform, and remain on it for 10 seconds. Mice were then removed and dried before their next run. Mice received three training sessions, with a 5 minutes break between session over the 5 days. The time until the mouse mounted the platform (escape latency) was recorded. A computer-assisted video tracking system (Noldus Ethovision XT) recorded and measured distanced travelled (cm) and time spent to find the platform (escape latency, s). For probe trials at day 6, mice were placed in the tank without the platform and given 80 seconds to explore the tank. Noldus Ethovision software tracked swim speed, total distance moved, and time spent in the target quadrant where the platform was previously located.

Adult cardiomyocytes isolation, cell shortening and Ca²⁺ measurements. Adult cardiomyocytes were isolated from WT and hTau mice by enzymatic digestion as previously described (Graham, 2013). Briefly, mouse heart was excised and placed in physiological saline solution to allow the beating heart to wash out the blood. The aorta was cannulated and the heart perfused with Tyrode solution (containing in mmol/l: 137 NaCl, 4 KCl, 1 MgCl2, 10 HEPES, 0.33 NaH2PO4, 10 glucose, 5 Taurine, pH7.4) followed by the same solution containing collagenase (Collagenase B and D—Roche) and proteases (Type XIV Sigma). The cells were then dissociated mechanically in the same Tyrode solution containing 5% BSA. Isolated cardiomyocytes were placed in a superfusion chamber on the stage of an inverted microscope, superfused with Tyrode solution (containing in mmol/l: 137 NaCl, 4 KCl, 1 MgCl₂, 10 HEPES, 0.33 NaH2PO4, 5.5 glucose, 1.2 CaCl₂), pH 7.4) and electrically stimulated with a biphasic pulse (5 Hz).

Cell shortening and rates of contraction and relaxation were recorded online with a video-edge detection system and data acquisition (IonOptix Corp, Milton, MA). The fluorescent Ca²⁺ indicator Fura-2-AM (Invitrogen) was used to measure intracellular Ca²⁺, as previously described (Del Monte, F., 1999). Fura2 excitation spectrum shifts between 340 nm (F₃₄₀) and 380 nm (F₃₈₀) with emission at 510 nm. Fluorescence ratio (R=F₃₄₀/F₃₈₀) directly correlates to the [Ca²⁺]ᵢ. Cell shortening and Ca²⁺ transients were analyzed using the IonWizard (world-wide-web at ionoptix.com/products/ionwizard) software.

Immunoblotting. For immunoblotting, frozen human tissue was lysate in using two extraction buffers: 1) Lysis buffer (Cell Signaling 9803) containing 1% non-ionic detergent Triton X-100, 20 mM Tris pH7.5, 150 mM NaCl, 1 mM EDTA, 1 mM EGTA, protease and phosphatase inhibitor cocktails (Roche) for the soluble fraction or 2) Lysis buffer containing 2% ionic detergent SDS, 10 mM Tris pH7.5, 10 mM NaF, 5 mM DTT, 2 mM EGTA for the insoluble aggregates. Mouse tissue was lysate with Cell Signaling lysis buffer. Protein concentration of tissue lysate preparations was measured using the Bradford method (60). Proteins were separated by gel electrophoresis using 4-20% gradient criterion TGX stain free gels (Bio-Rad). For the immunoreactions, the blots were incubated separately with antibodies anti total tau (E178 Abcam ab 32057), phospho Ser^{396/404} (gift from Dr. Abisambra), phospho Ser²⁶² (Thermo-Fisher 404-750G). The processed blots were imaged using the Bio-Rad Chemidoc imager.

Enzyme-Linked-Immuno-Sorbent Assay (ELISA). ELISA was performed independently in the two collaborative laboratories (Dr. del Monte and Dr. Kayed). Dr. Kayed's ELISA was performed as previously published (Castillo-Carranza, 2014a; 2020). In Dr. del Monte's Lab expression levels tau and tau-pSer396-404 were quantified using Invitrogen ELISA Novex kit (Kf1B0042 and KH1B7031), according to the manufacturer's instructions.

Immunofluorescence staining. Immunofluorescence staining and analysis of human and mouse brains, hearts and cardiomyocytes were also carried out independently in the laboratories of Drs. Kayed, del Monte and Norris as detailed below.
Dr. Kaved Laboratory: The staining protocol in the laboratory of Dr. Kayed was performed on paraffin embedded sections or 4% paraformaldehyde fixed cardiomyocytes as previously described (Sengupta and Kayed, 2017; Catillo-Carranza, 2017). Brain and Heart tissues were blocked in 0.5 ml of 5% NGS Serum in Phosphate Buffered Saline (PBS) Tween (PBST) for 1 h. The primary antibodies (TOMA; α-Sarcomeric Actin Ab #28052; NeuN Ab #ab177487) were incubated in this mixture overnight at 4° C., then washed three times in PBST (10 min each). Secondary antibody (ThermoFisher Scientific) was diluted 1:800 in 5% NGS/PBST, and tissue were incubated for 2 h at room temperature (RT). After a wash, the nuclei were stained with DAPI diluted at 1:10,000 in PBST (5 mg/ml stock solution) for 5 min. Two washes were then performed (10 min each, PBST then PBS). Coverslips were mounted on glass slides with 8 μl Prolong Gold Antifade mounting media. The slides were air dried inside a fume hood at RT (or stored at 4° C. until they were placed in a fume hood). Single frame images and Z-stacks for 3D rendering and orthogonal view acquisition were collected using a Keyence BZ-800 microscope with a Nikon 100× oil immersion objective and assessed with a BZ-X Analyzer. Integrated density and Pearson colocalization coefficient analysis have been performed using ImageJ FIJI (NIH) as previously described (Montalbano et al., 2020). As negative control, samples were not probed with primary antibodies but were subject to equivalent assays conditions.
Dr. del Monte Laboratory: The staining protocol in the laboratory of Dr. del Monte was performed on frozen sections as previously described (Albayram et al., 2017; 2019). For the frozen sections the primary antibodies used were tau oligomeric monoclonal antibody TOMA (provided by Dr. Kayed), oligomeric tau T22 polyclonal antibody (ABN454; EMD Millipore), recombinant total tau monoclonal antibody E178 (ab32057; Abcam), recombinant sarcomeric alpha actinin EP2529Y monoclonal antibody (ab68167; Abcam). The sections were incubated with primary antibodies overnight at 4° C. For double immunofluorescence staining, the sections were incubated with Alexa Fluor 488 or 594 conjugated isotype-specific secondary antibodies (Jackson Immuno Research) for 1 hour at room temperature. Manufacturer-supplied blocking buffer (Vector Laboratories) was used for each reaction. The sections were washed six times with PBS after each step. Labeled sections were visualized with a Zeiss confocal microscope. The gain of confocal laser was set at the level where there are no fluorescence signals including autofluorescence in sections without primary antibody, but with secondary antibody. Immunostaining images and their colocalization (Mander's overlap coefficient) were quantified using ImageJ FIJI (NIH) as described.
Dr. Norris Lab: Paraformaldehyde fixed cardiomyocytes were washed with 1×PBS, permeabilized for 5 minutes with 0.1% Triton X-100 diluted in 1×PBS, washed twice with 1×PBS and blocked with 1% Bovine Serum Albumin (BSA) in 1×PBS for 1 hour at room temperature. Cardiomyocytes were incubated overnight at 4° C. with the following primary antibodies: alpha-tubulin (Novus, #NB100-690SS) and RYR2 (Sigma Aldrich, #HPA020028). Following three 5-minute washes in 1×PBS, cells were incubated with secondary antibodies conjugated with Alexa Fluors 488 and Cy5 (Invitrogen) for 1 hour at room temperature, washed with 1×PBS including a 10 minutes incubation with Hoechst (Life Technologies, #H3569) diluted in 1×PBS, and cover-slipped with SlowFade Gold Antifade Reagent (Invitrogen, #S36936). Immunofluorescence was imaged with a M2 Leica TCS SP5 AOBS Confocal Microscope equipped with LAS AF v2.6.3 Build 8173 Acquisition and Analysis Software.

TOMA Antibody treatment. Twelve-month-old hTau mice were treated with either IgG (12 mice, 6 male/6 female) or anti-tau-PAO structural antibodies (TOMA) (12 mice, 6 male/6 female). Mice were treated with a single intraperitoneal injection of TOMA as previously described (Castillo-Carranza, 2014a; 2014b).

Statistical Analysis. Statistical analyses were performed as detailed below:
In-vitro human data were analyzed using non-parametric two-tailed Mann-Whitney test for the SDS page. The statistical analysis for the ELISA was performed in GraphPad Prism (version 9.0.1) using a two-way ANOVA with Bonferroni multiple comparison.
In-vivo echocardiography data were analyzed in R (Version 4.0.3) with either parametric t-test or nonparametric Mann-Whitney U test following appraisal of model assumptions. Genotype and age effects were determined in GraphPad Prism (Version 9.0.1) using two-way ANOVA with Bonferroni correction.
In-vitro isolated cardiomyocytes contractility and Ca²⁺ transient data were analyzed in R through use of a linear mixed effects model (LMM). Continuous variables from multiple cells were compared across WT and hTau groups, with each mouse serving as a clustering variable to account for random effects. Model assumptions were checked graphically and log 10 transformation was considered where appropriate, with data presented as either arithmetic or geometric means. Post-hoc pairwise comparison with P-value adjustment following the Holm-Bonferroni method was performed, and P-values less than 0.05 were deemed statistically significant.
   Behavior data were analyzed in GraphPad Prism (version 8.0.1) using a two-way ANOVA with Bonferroni correction (Morris water maze Escape Latency) or non-parametric two-tailed Mann-Whitney test (Morris water maze, Y maze and Open Field test).

Rigor and reproducibility. Rigor and reproducibility were enhanced by the collaborative nature of the study performed in three laboratories. Some experiments were duplicated and others performed blind in the different laboratories for different aspects. ELISA and IF were performed independently and blind in two participating laboratories, with mice injected in one laboratory and studied blind in the other laboratory. Samples were sent back to the first laboratory for IF. The IF for the mechanism was performed blind on isolated mouse cardiomyocytes.

### Example 2—Results

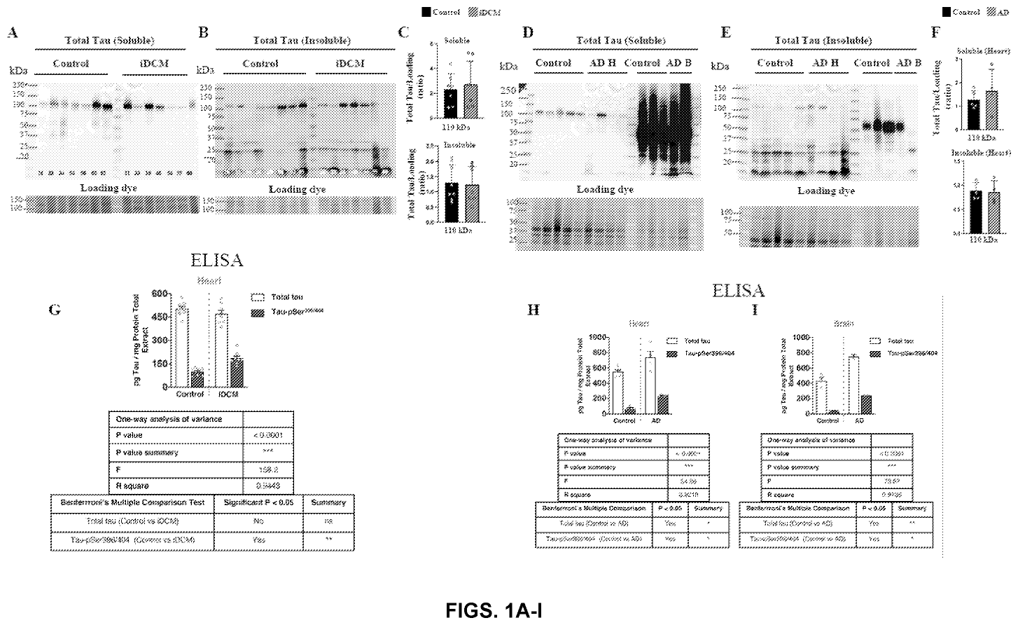

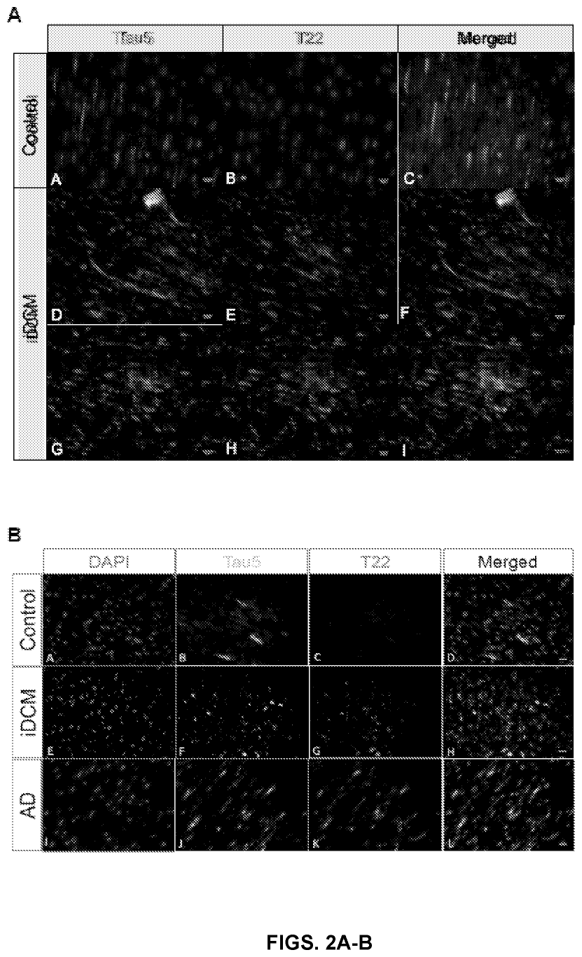

Big-tau is the human hearts isoform. The inventors tested heart samples from idiopathic dilated cardiomyopathy (iDCM) and non-failing age/sex/ethnicity matched controls as well as brain and heart tissue from four cases with medical history and pathological evidence of AD (Table S1) and matched controls (detailed in the methods section) (Troncone et al., 2016). The AD samples were previously used to describe that Aβ pathology affects the heart of AD patients (Troncone et al., 2016). These new data show that tau is expressed in human hearts (FIGS. 1A-I). Total tau expression by SDS page was not significantly different both in the iDCM and the AD groups (FIGS. 1A-E), whereas ELISA, using Tau-5, showed higher expression in both AD heart and brain (FIGS. 1H-I). The lack of significance in the SDS page can be due to the semi-quantitative nature of the method and the inter-patients variability also shown in AD brains (Sjogren et al., 2001). Additionally, iDCM is a diagnosis of exclusion and the diagnosis of iDCM includes a heterogeneous group of cardiomyopathies clustered by the dilated phenotype and not the underlying pathogenesis. Thus tauopathy (as other proteinopathies (Troncone et al., 2016; Taylor et al.) may occur in a subgroup of cases. Instead, ELISA demonstrated that tau is more expressed in the heart and in the brain of AD patients compared to controls (FIGS. 1H-I). Interestingly, the inventors found that the most represented isoform in the heart is the 100-110 kDa isoform, known as big-tau (Georgieff et al., 1993(Immunofluorescence showed that tau is expressed inside cardiomyocytes and co-localizes with PAO stained with the structural antibody against PAO (T22) in diseases (FIGS. 2A-B).

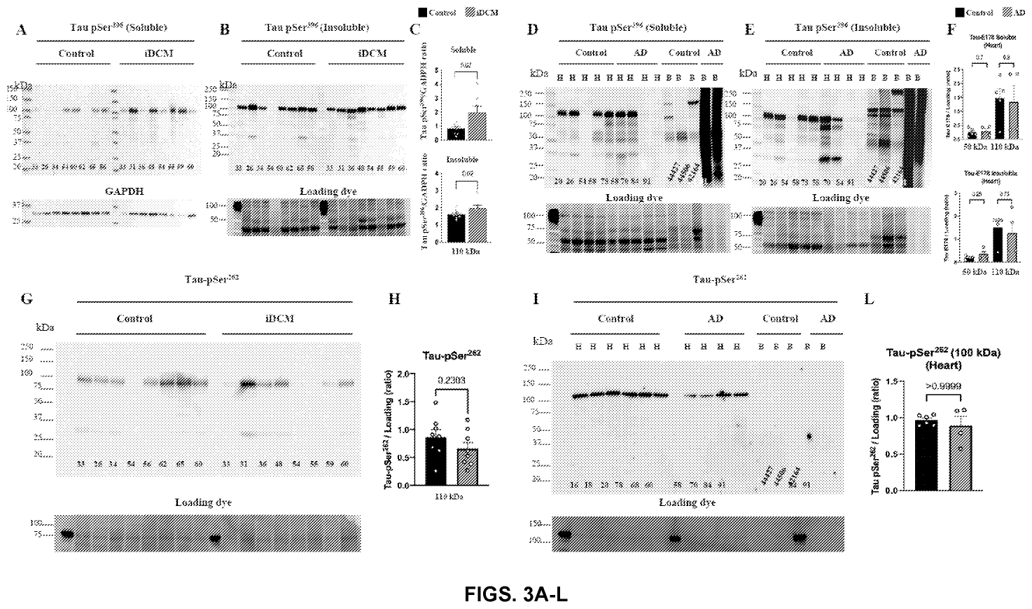

Tau phosphorylation and pathology in human hearts. Tau pathology is composed of paired helical filaments, which are primarily formed by hyper-phosphorylated tau, and tau p-Ser³⁹⁶ is traditionally associates with AD tangles (Taylor et al., 2007). As in the brain, the inventors found that tau is hyperphosphorylated on Ser³⁹⁶ both in the soluble and insoluble fractions in iDCM hearts (FIGS. 3A-C). p-Ser³⁹⁶ was not significantly increased in the heart of AD patients possibly due to the variability of the samples, whereas the brain signal couldn't be quantified due to saturation (FIGS. 3D-F). However increased tau p-Ser³⁹⁶ was shown by ELISA (FIGS. 1G-I).

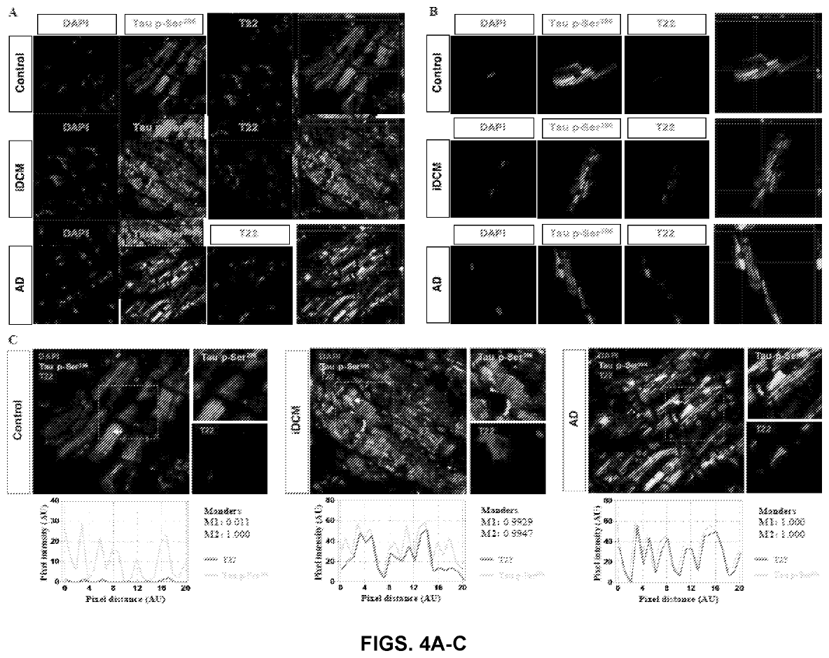

By immunofluorescence p-Ser³⁹⁶ colocalizes in the myocardium with the toxic tau-PAO stained with structural antibodies (T22) (FIG. 4A) and is present inside the cardiomyocytes in iDCM and AD, while they are undetectable in the control tissue (FIG. 4B).

Among all the numerous tau phosphosites, p-Ser²⁶² has been reported to be involved in tau aggregation (Wille et al., 1992). Different from p-Ser³⁹⁶, the inventors found that Ser²⁶² phosphorylation was decreased in iDCM hearts (although non-significantly) (FIGS. 3G-H). Furthermore, differently from other reports that found hyperphosphorylated tau p-Ser²⁶² in AD brains, in our study tau p-Ser²⁶² wasn't detectable in the human brains (FIG. 3E) and, although non-significant, p-Ser²⁶² tends to be reduced in AD hearts (FIGS. 31, 3L). Those data support the previously reported protective effect of this PTM against paired-helical filaments (PHF) assembly (Schneider et al., 1999).

Functional readout of cardiac tauopathy in mice. The inventors tested the pathogenic effect of cardiac tauopathy in a well-established transgenic mouse model of humanized tau (hTau). They performed the behavioral tests to compare the time course of the brain with cardiac pathology and phenotype in the same mice. As previously shown (Andorfer et al., 2003; Polydoro et al., 2009; Geizler et al., 2016; Phillips et al., 2011), cognitive performance was unaffected in the 3- and 7-months old hTau mice compared with C57Bl/6J age-matched controls. The spatial learning and memory tested by Morris water maze (Figs. S1A-B) and novelty Y-maze (Fig. S1C) became significantly impaired in the 12-months-old mice while the open field test showed no significant abnormalities for ages and genotypes (Fig. S1D). Using anti-Tau-Oligo-Monoclonal-Antibodies (TOMA), the inventors confirmed an age-dependent tau pathology in hTau mouse brain (Fig. S1E) (Andorfer et al., 2003; 2005).

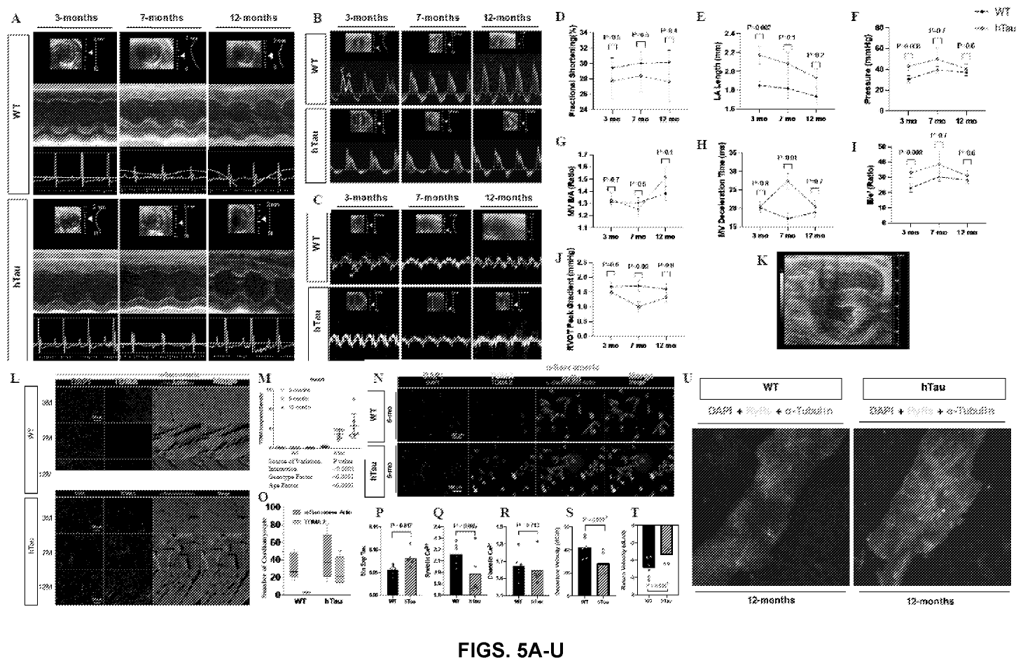

Different from the brain, progressive myocardial dysfunction was present from 3-month-old hTau mice by echocardiography (FIGS. 5A-J, Table S2, Fig. S2) accompanied by accumulation of tau-PAO (FIGS. 5L-M; Fig. S3). Systolic function was lower, but non-significant at all ages in hTau mice with the worse values at 12 months (FIG. 5D). Diastolic function appeared progressively impaired with age in hTau mice when parameters were compiled as defined by the degrees of diastolic dysfunction (Fig. S4A). E/A ratio (FIG. 5G) wasn't significantly different, however showed an averaged reduced value at 7-month and an increased value at 12-months of age supporting a progressive worsening of diastolic function from grade-II to grade-III, as also paralleled by the mirror changes in mitral valve deceleration time (FIG. 5H). The mean left-atrial (LA) pressure was steadily higher (FIG. 5F, Table 2), in 3- and 7-months-old hTau mice. The increased right ventricular (RV) pressure was severe in some mice where it caused paradox septal movement (FIG. 5K) impeding the accurate hemodynamic measurements of the left ventricular end diastolic pressure (LVEDP).

The in-vivo echocardiographic findings were confirmed in cardiomyocytes isolated from 6-months-old hTau mice and C57Bl/6J control hearts. Systolic function was lower, but non-significant, also in hTau cardiomyocytes (Table S3) whereas diastolic defect was confirmed as shown by i) stiffer sarcomeres revealed by higher tau value (FIG. 5P; Fig. S5, Table S3) and ii) abnormal Ca²⁺ homeostasis revealed by the reduced Ca²⁺ peak amplitude and the significant delayed rise and decay velocities (FIG. 5Q-T; Fig. S5; Table S3). An inhomogeneous accumulation of tau-PAO in the cardiomyocytes may explain the high variability of cardiomyocyte values (FIGS. 5N-O; Fig. S6).

Tau-PAO dual mechanisms for cardiac disease. For a long time, the accumulation of amyloid aggregates failed to correlate with cognitive deficit in AD (Chen et al., 2015; Chatrchyan et al., 2012), until was it found that PAO pathology better parallels the functional symptoms both in human and animal models. As detailed before, accumulation of tau-PAO was detectable in three-month-old hTau mice and progressively accumulated with age (FIGS. 5L-M) paralleling myocardial functional deficit.

PAO aggregates are known to affect Ca²⁺ homeostasis both in neurons and cardiomyocytes (Demuro & Parker, 2013; Demuro et al., 2010; Esteras et al., 2021; Gianni et al., 2010), potentially explaining the observed changes in Ca²⁺ handling in hTau cardiomyocytes. However, since tau functions by stabilizing microtubules that connect the nucleus to the sarcoplasmic reticulum in cardiomyocytes for the trafficking of Ca²⁺-handling proteins (Fig. S7), the inventors hypothesized that hyperphosphorylated tau would also affect Ca²⁺-homeostasis by impeding Ca²⁺-handling protein trafficking through microtubules disarray. By immunofluorescence, cardiomyocytes isolated from hTau mice showed disarray of the microtubule network corresponding to disorganized distribution of the Ca²⁺-handling proteins as exemplified by the disrupted distribution of RyRs (FIG. 5U).

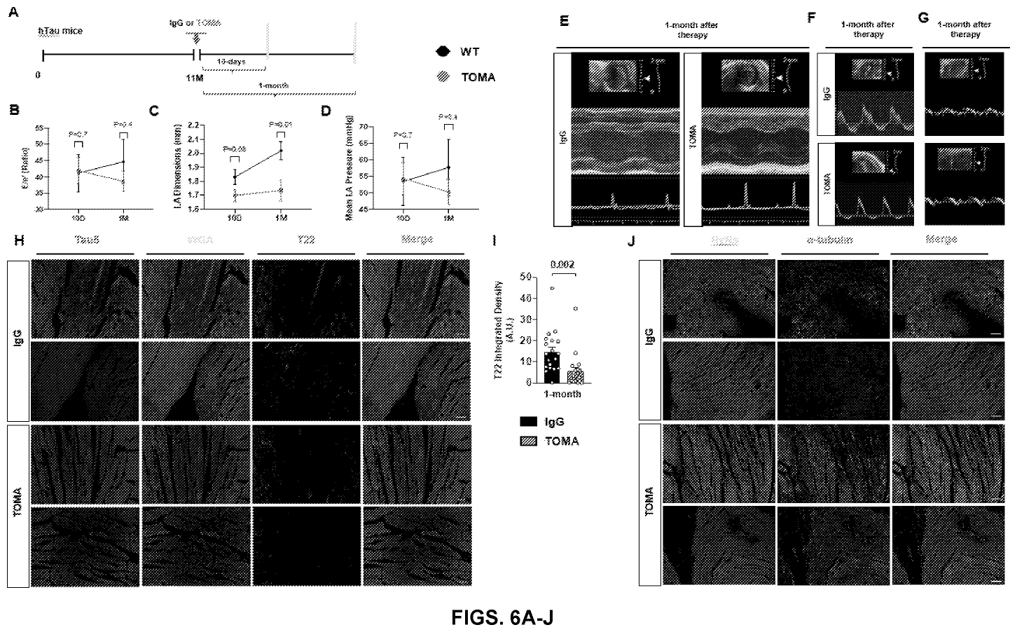

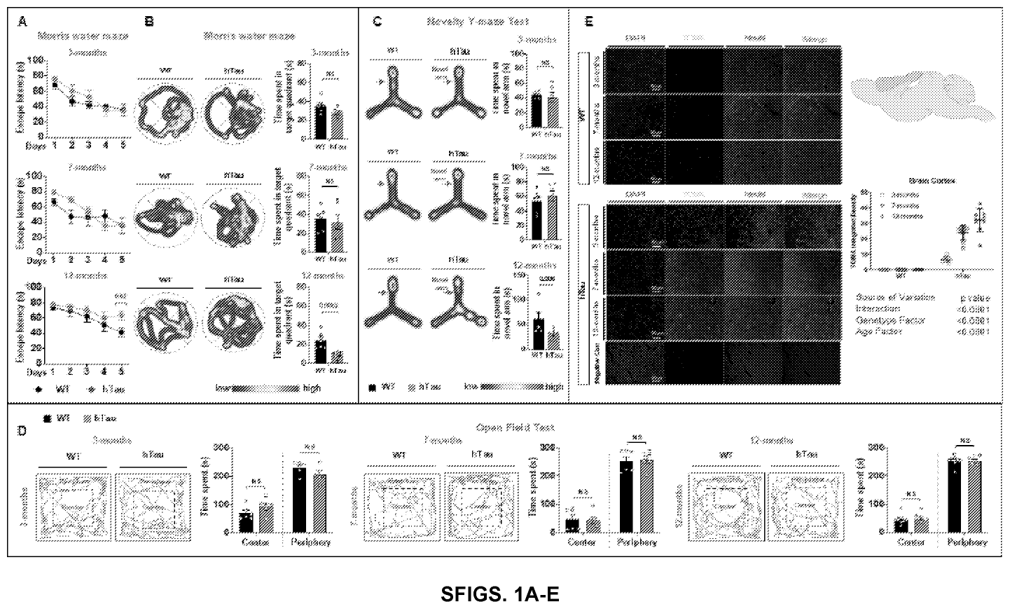

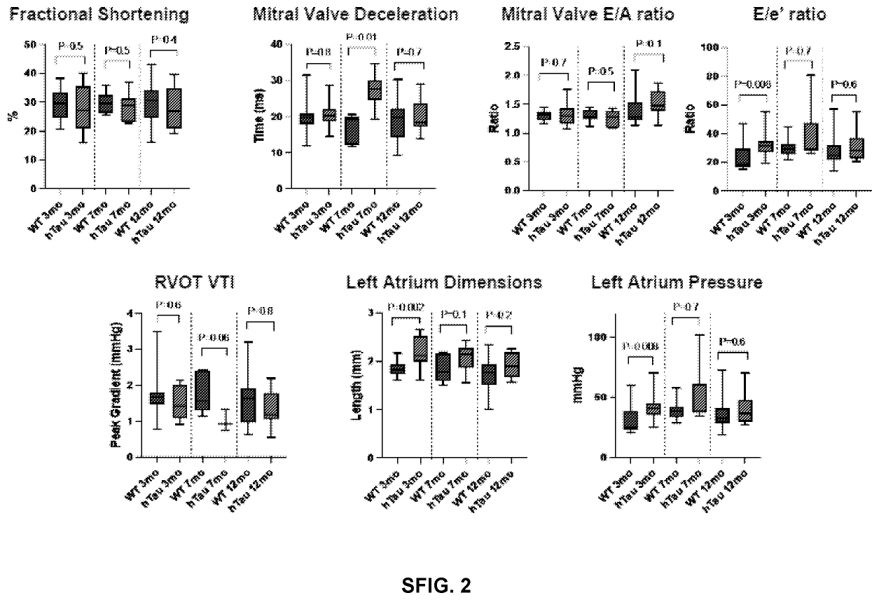

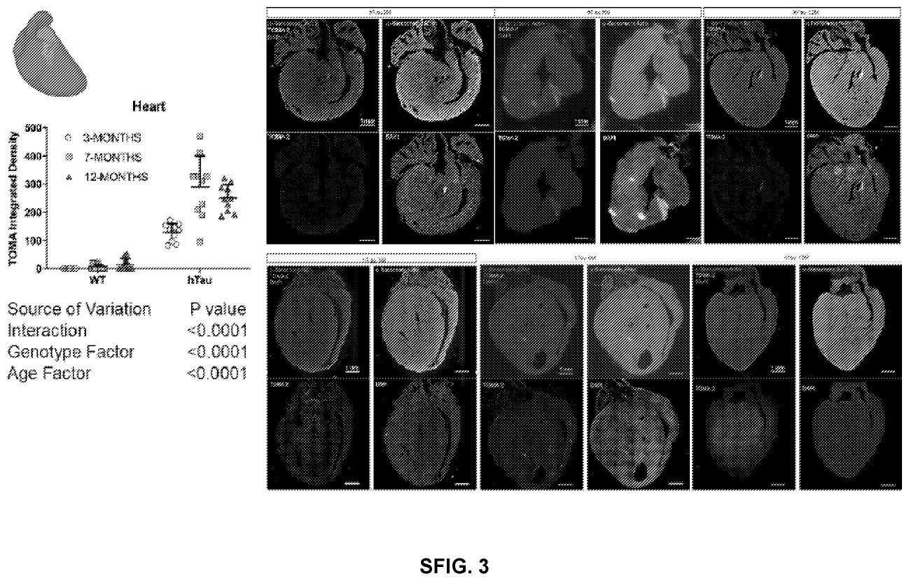

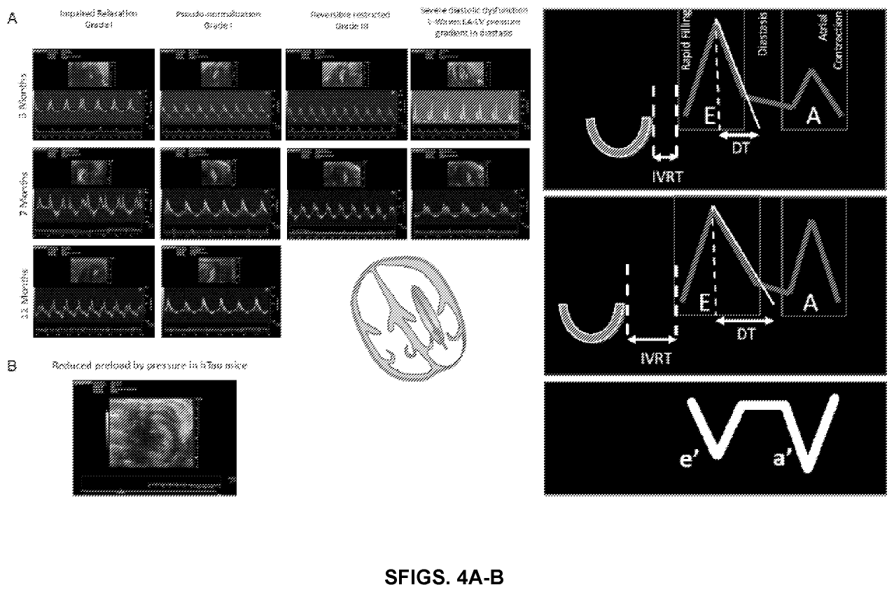

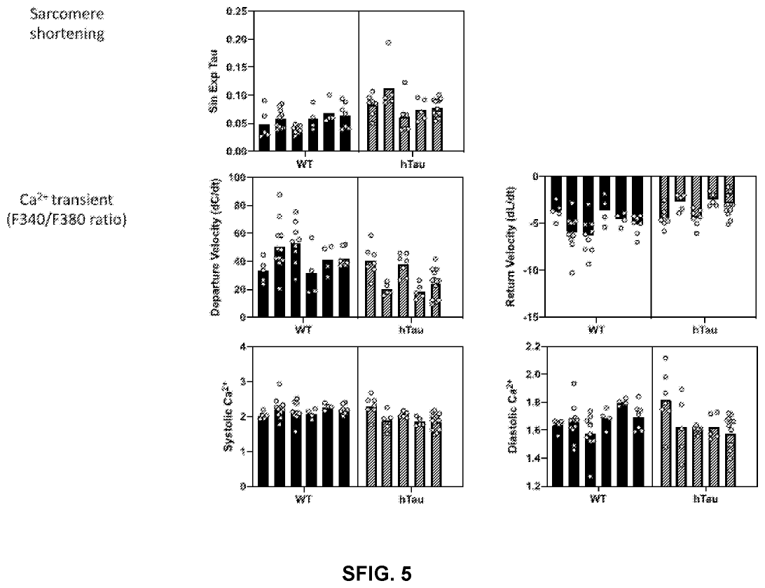

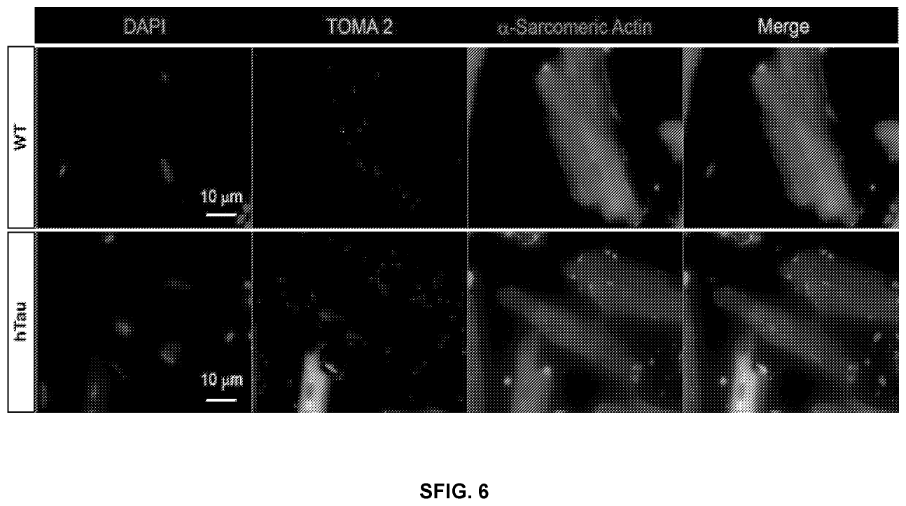

TOMA therapy for cardiac tauopathy. Monoclonal antibody (mAb) therapy is a recently introduced, and rapidly expanding, modality for the treatment of human diseases. To mimic the studies in the brain (Castillo-Carranza et al., 2014; 2015), same age male and female hTau mice were injected (once) intraperitoneally with TOMA or IgG control. Mice were studied 10 and 30 days after injection by echocardiography (FIG. 6A). Whereas no significant changes were detected at the 10-day time-point, 30-days after treatment diastolic function was improved as shown by the E/e′ ratio, mean LA pressure and a significant reduction in LA dimension (FIGS. 6B-D, 6F, 6G). Although the measurements of filling pressure were not significant, the pseudo-normalization in the most severe dysfunction might explain the result. The degree of dysfunction based on the algorithm that includes E/A ratio, E-wave deceleration time and LA dimension showed that a grade-IV diastolic dysfunction in >⅓ of IgG-treated mice compared to TOMA-treated mice where the majority of mice (>⅓) showed grade I/II. The functional improvement was accompanied by a reduction in the accumulation of tau-PAO (FIGS. 6H, 6J), and a significant remodeling of the transverse tubule architecture corresponding to a more ordered distribution of Ca²′ handling proteins as simplified by the RyRs (FIG. 6I), setting the first evidence of anti-proteotoxic mAb therapy for HF.

### Example 3—Discussion

Evidence that misfolded protein aggregates are not isolated to the brain of AD patients, but affect other organs (Wang et al., 2017) is steadily emerging with the discovery of amyloid deposits beyond the brain, in the skeletal muscle (Kuo et al., 2000), guts (Joachim et al., 1989) and skin (Wang et al., 2017; Akerman et al., 2019). The inventors described amyloid deposits in HF and AD hearts in human (Gianni et al., 2010) and animal models (Sanbe et al., 2004; Subramanian et al., 2015), associated with progressive age-dependent HF with preserved ejection fraction (HFpEF) and Aβ fibrillar and non-fibrillar pre-amyloid-oligomers (PAO) (Troncone et al., 2016) accumulation. This novel finding supports the pathological link between AD and HF (a long-known significant risk factor for AD) besides the most commonly acknowledged perfusion defects (Iadecola, 2004) or abnormal vascular reactivity (Iturria-Medina et al., 2016). The inventors now describe that tau and tau aggregates are present in the human heart in normal and disease, strengthening this connection.

While Aβ has been documented outside the brain, tau has been traditionally considered a brain-specific protein and whether tau plays a role also in the pathogenesis of HF, and by which mechanism(s) is unknown. The idea that tau is expressed and involved in the pathogenesis of diseases outside the brain has been long neglected despite a) the demonstrated expression of MAPT in several organs (including the heart); b) rodent proteomics suggesting that tau is also expressed in the myocardium (Drastichova et al., 2012); and c) the myocardial hypertrophic phenotype that results from knocking-down the gene in rodents (Betrie et al., 2017). Moreover, tubulin (stabilized by tau to form microtubules), is a major component of the cytoskeleton in eukaryotes, preserving cardiomyocyte's integrity and function. In fact, tubulin is essential for intracellular trafficking of vesicles including the ones carrying Ca²¹-handling proteins, key to cardiomyocytes contractility. Notably, a significant pathogenic aspect of tau in cardiomyocytes is that 30% of total tubulin is polymerized as microtubules, and 70% occurs as non-polymerized cytosolic protein (Tagawa et al., 1998), providing low margin for reserve. This may underlie an increased pathogenic susceptibility of the heart and implicates a compelling role in the heart of the emerging atypical functions of tau (Sotiropoulos et al., 2017).

Another atypical aspect of myocardial tau is that the predominant isoform is the 100-110 kDa, known as big-tau (Georgieff et al., 1993) (FIGS. 1A-I). Tau is expressed as multiple isoforms ranging from 45 to 110 kDa from alternative splicing of the MAPT gene, the presence of a different number of repeats of the microtubule-binding domain and the expression/lack of exons 4 and 6. Big-tau, formerly known as high molecular weight (HMW) tau, was described the adult peripheral nervous system (PNS) (Drubin et al., 1988; Boyne et al., 1995; Nothias et al., 1995). It is encoded by an 8 kb transcript of exon-4a with/without the expression of exon-6. Interestingly. Interestingly, it has been shown that the extra 250aa added to the N-terminal of the protein (Fischer & Baas, 2020) increase the microtubules spacing lowering resistance and reducing the energy of transport. It was also shown that big-tau separates the phosphatase activity domain (PAD) from the microtubule-motor interface releasing the obstacle to organelles transport, also important for Ca²⁺ handling proteins recruitment under stress and potentially increasing contractile efficiency (Fischer Bass, 2020). In the myocardium, these would result in a more efficient Ca²⁺ handling proteins recruitment under stress and contractile efficiency (Fischer & Bass, 2020).

Since tau is highly soluble, its aggregation and polymerization should be unlikely. However, even with low efficiency, the tau microtubule-binding repeat domains can facilitate the formation of PHF (Wille et al., 1992 (which contain aggregates of hyperphosphorylated tau. Physiological phosphorylation acts as a key regulatory mechanism in inhibiting tau's ability to bind microtubules, impairing their stability. When pathological, tau hyperphosphorylation alters protein stability promoting aggregation and NFT formation (Kosik, 1993). As in the brain, the inventors found that tau is hyperphosphorylated on Ser³⁹⁶ (which is a marker of mid-stage tau tangles in the brain) (Augustinack et al., 2002) also in diseased hearts (FIGS. 1G-H, FIG. 3A-C). Phosphorylated tau was also significantly more expressed in the insoluble fraction (FIGS. 3A-C) and colocalizes with PAO in diseased cardiomyocytes (FIG. 4A-C) suggesting that phosphorylated big-tau is also prone to aggregation. These findings argue against the idea of expanding the size of tau as a therapeutic approach to prevent toxic misfolding, based on the assumption that big-tau has a lower propensity to polymerize in fibrils (Avila, 2000), proposed to explain the lower impact of tauopathy in the PNS.

Instead, p-Ser²⁶² was lower in the diseased hearts (FIGS. 3G-H) and undetectable in the human brains (FIGS. 31, 3L). Ser²⁶² is positioned in the first repeat, a region that forms the backbone of PHF, and has been reported to strongly reduce the affinity of tau for microtubules, indicating that hyperphosphorylation on this site should favor tau aggregation. p-Ser²⁶² was also reported to be required for the pathogenic interaction between Aβ₄₂ and tau in Drosophila (Iijima-Ando & Iijima, 2010) and rats (Majd et al., 2017), potentially driving aggregation. In our study, decreased Ser²⁶² phosphorylation points against a role of this phosphorylation in aggregation. Accordingly, p-Ser²⁶² was found to inhibit tau's assembly in NFT (Schneider et al., 1999) and only weak phosphorylation on this site was previously reported in human brains with minor progression with increased in Braak stage, and absence of p-Ser262 was shown to drive tau aggregation in vitro (Despres et al., 2017). Altogether these discrepancies call for further studies on the significance of this PTM in the onset and progression of brain and heart diseases. This is important since p-Ser²⁶² is the target of protein kinase A, configuring this pathway as a novel therapeutic route, already considered for HF (Saad et al., 2020).

Overall, the human data provide indirect evidence that big-tau may contribute to myocardial dysfunction since in its hyper-phosphorylated form is sequestered in the aggregates both in iDCM and AD (FIGS. 3A-C, FIGS. 4A-C). However, although suggestive, the human samples do not establish a direct link between tau pathology and functional changes in the brain and heart. Furthermore, while AD hearts were not in end-stage HF showing the HFpEF phenotype as previously shown (Troncone et al., 2016), the iDCM hearts were collected from explanted hearts, when the patient had developed HFrEF. However, although HFpEF and HFrEF have recognized different pathophysiological origin, the two may coexist (Schiattarella et al., 2020) and systolic function may gradually decline with some HFpEF evolving into HFrEF (Sanderson, 2016). Additionally, while chronic cerebral hypoperfusion has been considered the main factor contributing to dementia, HFpEF, but not HFrEF has been associated with dementia. Hence, proteinopathy may represent a novel underlying cause of HFpEF in addition to traditional hypertension and metabolic syndrome.

Thus, the inventors tested the age-dependent molecular-to-functional link in hTau mice. In this model, they linked tau pathology to functional changes in male and female hTau mice at three ages. While the functional deficit in the brain was delayed compared to PAO accumulation (Fig. S1), myocardial dysfunction (FIGS. 5A-J; Fig. S2, Fig. S4; Table S2) appeared as soon as PAO deposit. As in AD patients (Troncone et al., 2016), hTau mice showed diastolic disfunction as defined by the algorithm used to define HFpEF in human in-vivo and at the single cell level. Furthermore, as in AD patients (Troncone et al., 2016), cardiac function in WT mice progressively approaches the age matched myocardial dysfunction in hTau, reinforcing the concept that AD is an early aging phenotype also in the heart.

Whether Aβ-PAO accumulation is a known mechanism causing Ca²⁺ dishomeostasis in neurons (Demuro & Parker, 2013) and cardiomyocytes (Gianni et al., 2010), and tau-PAO have been shown to disrupt Ca²⁺ homeostasis in neurons (Shafiei et al., 2017), tau pathology may contribute to Ca²⁺ dishomeostasis also by altering the cytoskeletal architecture leading to disarray of microtubules network and disrupting Ca²⁺ handling protein trafficking and distribution (FIG. 5U). This finding provides a novel mechanism for the RyRs-LTCC uncoupling known to occur in HF (Kolstad et al., 2018) and may serve as novel therapeutic target to improve Ca²⁺ homeostasis and function in HF, as shown by our treatment data.

Monoclonal antibody (mAb) therapy is a rapidly expanding modality for the treatment of human diseases. However, possibly due to the lack of a single target and the recent discovery of pathological aggregates in the heart, anti-PAO immunotherapy was never tested for HF. Here, the inventors show that anti tau-PAO antibody treatment (TOMA) reduces tau-PAO aggregates in the heart (FIGS. 6H-I) and improves diastolic function one month after therapy (FIGS. 6B-G; Table S4). TOMA treatment acting of both arms of the dual pathogenic mechanisms of tauopathy sets the first evidence for anti-proteotoxic immunotherapy for HF.

TABLE S1
Clinical Parameters of iDCM, AD and respective controls
included in the study

| | | | | | Associated | | FS/ |
|---|---|---|---|---|---|---|---|
| | Type | Age | Sex | Ethnicity | Diseases | HW | EF |
| 1 | Control | 26 | F | Caucasian | Chemotherapy | 240 | 25 |
| | | | | | | | (FS) |
| 2 | Control | 33 | M | Caucasian | Depression, SKZ | 450 | 35 |
| 3 | Control | 64 | F | Caucasian | ETOH, Liver | NA | NA |
| | | | | | cirrhosis | | |
| 4 | Control | 54 | F | Caucasian | Depression, DA | NA | NA |
| 5 | Control | 56 | M | Caucasian | | 630 | 76 |
| 6 | Control | 60 | M | Causasian | HTN | NA | 60 |
| 7 | Control | 62 | F | African | HTN | 420 | 60 |
| | | | | American | | | |
| 8 | Control | 65 | M | Unknown | MVP | 475 | NA |
| 9 | iDCM | 31 | F | African | Asthma, | 376 | 10 |
| | | | | American | Pneumonia, | | (FS) |
| | | | | | HypoK | | |
| 10 | iDCM | 33 | F | Caucasian | PE, PHTN, OB, | 590 | 23 |
| | | | | | ETOH, DA, | | (FS) |
| | | | | | KD, NSVT | | |
| 11 | iDCM | 36 | M | Unknown | CRI, HTN, | 450 | 41 |
| | | | | | IDDM, KD | | |
| 12 | iDCM | 48 | M | Asian | AFI, hTyr, MR, | 393 | 12 |
| | | | | | PFO, VT | | |
| 13 | iDCM | 54 | M | Caucasian | HTN | 830 | 12 |
| | | | | | | | (FS) |
| 14 | iDCM | 55 | F | Caucasian | AF, DM, Goiter, | 250 | 22 |
| | | | | | KD, HThy, | | (FS) |
| | | | | | HPThy, | | |
| | | | | | PHTN, VT | | |
| 15 | iDCM | 59 | M | Caucasian | CAD, Gout, | 710 | 30 |
| | | | | | HTN, HChol, | | (FS) |
| | | | | | hThy, Psoriasis, | | |
| | | | | | Stroke | | |
| 16 | iDCM | 60 | M | Caucasian | AF, ICD | NA | 14- |
| | | | | | | | 17 |
| 1 | Control | 16 | F | Csuossian | ADHD | 214 | 30 |
| | | | | | | | (FS) |
| 2 | Control | 18 | F | Caucasian | | 220 | NA |
| 3 | Control | 20 | M | Asian | | NA | 67 |
| 4 | Control | 26 | F | Caucasian | Chemotherapy | 240 | 25 |
| | | | | | | | (FS) |
| 5 | Control | 54 | F | Caucasian | Depression | NA | NA |
| 6 | Control | 58 | F | Caucasian | Depression, HTN | 270 | 64 |
| | | | | | | | (FS) |
| 7 | Control | 70 | F | Caucasian | HChol | NA | NA |
| 8 | Control | 78 | M | Caucasian | | NA | NA |
| 9 | AD | 58 | F | Caucasian | | 304 | NA |
| 10 | AD | 70 | F | Caucasian | NIDDM, hThy | 313 | NA |
| 11 | AD | 84 | | Unknown | ASDH, Anemia, | NA | NA |
| | | | | | Depression, | | |
| | | | | | GERD HChol, | | |
| | | | | | HTN, KF, | | |
| | | | | | Stroke | | |
| 12 | AD | 91 | M | Caucasian | HTN, NIDDM, | NA | NA |
| | | | | | Stroke | | |

Abbreviations: ADHD = Attention deficit hyperactivity disorder;
AF = Atrial fibrillation;
AFl = Atrial flutter;
AVBIII = 3rd degree Atrio-Ventricular Block;
CAD = Coronary Artery Disease;
CRI = Chronic renal insufficiency;
DA = drug abuse;
DM = Diabetes Mellitus;
ETOH = alcoholism;
HChol = hypercholesterolemia;
HypoK = hypokalemia;
HPTyr = hyperparathyroidism;
hThy = Hypothyroidism;
Hthy = hyperthyroidism;
HTN = Hypertension;
ICD = Intra cardiac defibrillator;
iDCM = idiopathic dilated cardiomyopathy;
IDDM = insulin dependent diabetes mellitus;
KD = Kidney Disease;
KF = kidney failure;
MR = mitral valve regurgitation;
MVP = mitral valve prolapse;
NIDDM = non-insulin dependent diabetes mellitus;
NSVT = non-sustained ventricular tachycardia;
OB = Obesity;
PE = Pulmonary embolism;
PFO = Patent Foramen Ovale;
PHTN = Pulmonary Hypertension;
PMK = pacemaker;
SKZ = schizophrenia;
VT = Ventricular Tachycardia.
For the HF patients cardiac function was recorded either as ejection fraction (EF) or fractional shortening (FS).

TABLE S2
Echocardiographic parameters of hTau and C57BI/6J mice at 3, 7 and 12
months of age

| 3 Months | | WT | | hTau | | |
|---|---|---|---|---|---|---|
| SAX | | AVG | SD | AVG | SD | F |
| Heart Rate | BPM | 465.509 | 59.878 | 441.165 | 54.850 | 0.200 |
| LVEDs/TL | Ratio | 0.149 | 0.0170 | 0.155 | 0.0359 | 0.567 |
| LVEDd/TL | Ratio | 0.212 | 0.0158 | 0.209 | 0.0292 | 0.795 |
| Volume; s | uL | 25.856 | 9.125 | 28.796 | 16.634 | 0.792 |
| Volume; d | uL | 58.725 | 12.523 | 57.874 | 21.770 | 0.900 |
| Stroke Volane | uL | 32.869 | 5.724 | 29.078 | 7.523 | 0.149 |
| Ejection Fraction | % | 56.881 | 7.738 | 53.788 | 13.115 | 0.480 |
| Fractional Shortening | % | 28.440 | 5.010 | 27.718 | 8.205 | 0.532 |
| Cardiac Output | mL/min | 15.399 | 3.779 | 13.021 | 4.386 | 0.143 |
| LV Mass | mg | 85.529 | 20.287 | 92.907 | 30.815 | 0.461 |
| LV Mass Corrected | mg | 68.423 | 16.230 | 74.326 | 24.652 | 0.461 |
| LVAW; s | mm | 1.062 | 0.140 | 1.018 | 0.154 | 0.440 |
| LVAW; d | mm | 0.690 | 0.115 | 0.752 | 0.107 | 0.164 |
| LVPW; s | mm | 1.028 | 0.0997 | 1.024 | 0.118 | 0.929 |
| LVPW; d | mm | 0.689 | 0.0628 | 0.733 | 0.105 | 0.277 |

MV

| | | | | | | |
|---|---|---|---|---|---|---|
| ET | ms | 47.283 | 5.726 | 43.887 | 4.604 | 0.108 |
| IVCT | ms | 18.651 | 3.316 | 17.818 | 3.216 | 0.347 |
| IVRT | ms | 20.476 | 4.175 | 20.450 | 4.434 | 0.987 |
| MV Deceleration | mm/s2 | −39520.164 | 18025.927 | −32765.223 | 7896.793 | 0.486 |
| MV Deceleration | ms | 20.135 | 5.249 | 20.669 | 3.829 | 0.771 |
| MV E/A | Ratio | 1.310 | 0.0922 | 1.330 | 0.205 | 0.739 |

TDV

| | | | | | | |
|---|---|---|---|---|---|---|
| IVCT | ms | 20.526 | 3.406 | 18.800 | 4.883 | 0.326 |
| IVRT | ms | 21.878 | 3.936 | 21.472 | 6.562 | 0.544 |
| E/E' | Ratio | 22.992 | 9.499 | 32.949 | 10.444 | 0.008 |

PA

| | | | | | | |
|---|---|---|---|---|---|---|
| RVOT VII | mmHg | 1.684 | 0.590 | 1.514 | 0.460 | 0.567 |

LA

| Length | mm | 1.851 | 0.158 | 2.170 | 0.319 | 0.002 |
|---|---|---|---|---|---|---|
| Pressure | mmHg | 30.525 | 11.827 | 42.921 | 13.003 | 0.008 |
| 7 Months | | WT | | hTau | | |
| SAX | | AVG | SD | AVG | SD | P |
| Heart Rate | BPM | 472.893 | 19.295 | 429.451 | 32.420 | 0.012 |
| LVEDs/IL | Ratio | 0.156 | 0.0138 | 0.156 | 0.0173 | 0.964 |
| LVEDd/TL | Ratio | 0.223 | 0.0128 | 0.2170 | 0.0169 | 0.499 |
| Volume; s | uL | 31.237 | 6.646 | 29.273 | 7.061 | 0.616 |
| Volume; d | uL | 73.358 | 10.133 | 64.896 | 10.184 | 0.234 |
| Stroke Volume | uL | 42.121 | 6.260 | 35.623 | 6.658 | 0.097 |
| Ejection Fraction | % | 57.580 | 5.700 | 55.100 | 7.921 | 0.526 |
| Fractional Shortening | % | 30.030 | 3.837 | 28.370 | 5.233 | 0.523 |
| Cardiac Output | mL/min | 19.970 | 3.404 | 15.270 | 2.825 | 0.021 |
| LV Mass | mg | 112.507 | 17.285 | 104.071 | 17.608 | 0.403 |
| LV Mass Corrected | mg | 90.006 | 13.828 | 83.257 | 14.087 | 0.403 |
| LVAW; s | mm | 1.203 | 0.112 | 1.135 | 0.102 | 0.281 |
| LVAW; d | mm | 0.763 | 0.0799 | 0.755 | 0.105 | 0.877 |
| LVPW; s | mm | 1.114 | 0.106 | 1.056 | 0.239 | 0.628 |
| LVPW; d | mm | 0.749 | 0.108 | 0.765 | 0.190 | 0.850 |

MV

| | | | | | | |
|---|---|---|---|---|---|---|
| ET | ms | 44.404 | 3.440 | 53.006 | 7.786 | 0.023 |
| IVCT | ms | 16.565 | 5.466 | 21.140 | 7.233 | 0.295 |
| IVRT | ms | 18.353 | 4.595 | 25.181 | 5.445 | 0.022 |
| MV Deceleration | mm/s2 | −50869.846 | 14237.807 | −26549.439 | 3556.465 | 0.001 |
| MV Deceleration | ms | 17.160 | 3.778 | 27.350 | 4.964 | 0.014 |
| MV E/A | Ratio | 1.304 | 0.118 | 1.252 | 0.147 | 0.486 |

TDV

| | | | | | | |
|---|---|---|---|---|---|---|
| IVCT | ms | 17.274 | 2.831 | 19.0546 | 3.517 | 0.333 |
| IVRT | ms | 19.442 | 3.754 | 17.704 | 4.758 | 0.477 |
| E/E' | Ratio | 30.210 | 7.335 | 38.530 | 20.880 | 0.731 |

PA

| | | | | | | |
|---|---|---|---|---|---|---|
| RVOT VTI | mmHg | 1.720 | 0.513 | 1.006 | 0.307 | 0.059 |

LA

| Length | mm | 1.819 | 0.279 | 2.081 | 0.300 | 0.138 |
|---|---|---|---|---|---|---|
| Pressure | mmHg | 39.510 | 9132 | 49.870 | 26.000 | 0.731 |
| 12 Months | | WT | | hTau | | |
| SAX | | AVG | SD | AVG | SD | P |
| Heart Rate | BPM | 453.563 | 74.960 | 414.372 | 51.156 | 0.172 |
| LVEDs/TL | Ratio | 0.143 | 0.0263 | 0.158 | 0.0308 | 0.176 |
| LVEDd/TL | Ratio | 0.206 | 0.0243 | 0.217 | 0.0229 | 0.269 |
| Volume; s | Ratio | 26.429 | 11.764 | 30.060 | 14.561 | 0.561 |
| Volume; d | uL | 60.452 | 15.816 | 62.121 | 16.706 | 0.802 |
| Stroke Volvose | uL | 34.023 | 7.364 | 32.061 | 5.484 | 0.488 |
| Ejection Fraction | uL | 57.690 | 10.140 | 53.570 | 11.820 | 0.356 |
| Fractional Shortening | % | 30.160 | 6.594 | 27.610 | 7.699 | 0.378 |
| Cardiac Output | % | 15.280 | 3.474 | 13.160 | 2.189 | 0.110 |
| LV Mass | mL/min | 97.375 | 18.909 | 99.224 | 29.428 | 0.844 |
| LV Mass Corrected | mg | 77.900 | 15.127 | 79.379 | 23.543 | 0.844 |
| LVAW; s | mg | 1.197 | 0.203 | 1.092 | 0.240 | 0.246 |
| LVAW; d | mm | 0.786 | 0.107 | 0.761 | 0.166 | 0.630 |
| LVPW; s | mm | 1.069 | 0.159 | 0.980 | 0.157 | 0.177 |
| LVPW; d | mm | 0.723 | 0.107 | 0.731 | 0.153 | 0.868 |

MV

| | | | | | | |
|---|---|---|---|---|---|---|
| ET | ms | 45.848 | 6.594 | 48.130 | 5.372 | 0.378 |
| IVCT | ms | 19.871 | 7.903 | 16.565 | 5.832 | 0.278 |
| IVRT | ms | 22.889 | 7.049 | 22.090 | 3.791 | 0.755 |
| MV Deceleration | mm/s2 | −40021.661 | 18224.507 | −32984.875 | 6309.869 | 0.463 |
| MV Deceleration | ms | 18.940 | 5.398 | 20.470 | 4.707 | 0.705 |
| MV E/A | Ratio | 1.381 | 0.252 | 1.518 | 0.228 | 0.131 |

TDV

| | | | | | | |
|---|---|---|---|---|---|---|
| IVCT | ms | 23.026 | 4.568 | 23.276 | 2.158 | 0.852 |
| IVRT | ms | 23.165 | 8.005 | 24.187 | 5.380 | 0.735 |
| E/E' | Ratio | 28.230 | 10.410 | 30.900 | 10.980 | 0.597 |

PA

| | | | | | | |
|---|---|---|---|---|---|---|
| RVOT VTI | mmHg | 1.602 | 0.787 | 1.324 | 0.504 | 0.792 |

LA

| | | | | | | |
|---|---|---|---|---|---|---|
| Length | mm | 1.736 | 0.353 | 3.933 | 0.259 | 0.195 |
| Pressure | mmHg | 37.048 | 12.961 | 40.380 | 13.670 | 0.597 |

Abbreviations: SAX = M Mode short axis measurement;
LVEDd/TL-Left Ventricular Dimension in diastole corrected for tibia length;
LVEDs/TL = Left Ventricular Dimension in systole corrected for tibia length;
LVAW; s = Left ventricular anterior wall thickness in systole;
LVAW; d = Left ventricular anterior wall thickness in diastole;
LVPW; s = Left ventricular posterior wall thickness in systole;
LVPW; d = Left ventricular posterior wall thickness in diastole;
MV = mitral valve;
AET = Aortic Ejection Time;
IVCT = isovolumetric contraction time;
IVRT = isovolumetric relaxation time;
MV E/A = Mitral Valve ratio of E = blood flow peak velocity from left ventricular relaxation in early diastole to A = atrial contraction flow peak velocity in late diastole;
TDV = Tissue Doppler Velocity;
E/e' = early mitral inflow velocity to mitral annular early diastolic velocity ratio;
RVOT VTI = Right Ventricular outflow tract velocity time integral;
LA = Left Atrium.
C57B1/6J wild-type (WT) mice: 6 male at 3 months, 7 male at 7 months, and 6 male/12 female at 12 months of age;
hTau mice: 6 male at 3 months, 6 male at 7 months, and 6 male/3 female at 12 months of age.

TABLE S3
Isolated cardiomyocyte measurements of cell shortening, Ca²⁺ transient and
sarcomere shortening

| | Cell Shortening | | P | Calcium Ratio | | P | Sorcomere Shortening | | P |
|---|---|---|---|---|---|---|---|---|---|
| | WT | hTau | adjusted | WT | hTau | adjusted | WT | hTau | adjusted |
| Baselien (bi) | — | — | — | | | 0.713 | — | — | — |
| Departure velocity (dep v) | | | | | | | | | |
| Departure velocity time (deg vel) | 0.022 | 0.022 | 0.919 | | | | | | 0.882 |
| Peak | — | — | — | 2.16 | 1.99 | | — | — | — |
| Baseline to % peak insight (bl%pekh) | | | | — | — | — | | | |
| Return velocity | | | | | | | | | |
| Mature velocity time | | | | 0.074 | | | | | |
| Time to baseline 50% | 0.097 | | 0.379 | 0.092 | | 0.821 | 0.097 | 0.105 | 0.270 |
| Sin exp tau | 0.066 | 0.080 | 0.200 | 0.124 | 0.122 | 0.375 | 0.086 | 0.082 | 0.017 |

```
Isolated adult cardiomyocytes contractile and Ca2+ parameters analyzed with Linear Mixed Model.
  values presented are geometric means obtained following back-transformation of log10 transformed data.
^{ϕ} p values correspond to log 10 transformed data-set.
Dep Vel = Departure Velocity-maximal rate of change during the contraction or calcium release phase of the transient;
bl%peakh = baseline length as a percentage of peak height is the baseline percent change during the transient;
Ret Vel = maximal rate of change during relaxation or calcium reuptake phase of the transient;
Time to bl 50% = The time to 50th percentage of the baseline.
Is a characterization of cellular relaxation or calcium reuptake; Sin exp tau = Single Exponential Tau is the exponential decay time constant. It characterizes the speed of relaxation recovery or Ca2+ reuptake. 8 male/2 female C57B1/6J wild-type (WT) and 6 male hTau mice were studies at 6 month of age. Data were analyzed in R through use of a linear mixed effects model (LMM).
  values presented are geometric means obtained following back-transformation of log10 transformed data;
^{ϕ} p values correspond to log 10 transformed data-set.
indicates data missing or illegible when filed
```

TABLE S4
Echocardiographic parameters of 12-month hTau injected IgG control or

| 10 Days | | IgG | | TOMA | | |
|---|---|---|---|---|---|---|
| SAX | Units | AVG | SD | AVG | SD | P |
| Heart Rate | BPM | 449.728 | 182.014 | 471.461 | 172.223 | 0.260 |
| LVEDs/TL | Ratio | 0.135 | 0.0211 | 0.146 | 0.0247 | 0.288 |
| LVEDd/TL | Ratio | 0.189 | 0.0248 | 0.197 | 0.0222 | 0.415 |
| Volume; s | uL | 22.294 | 10.349 | 26.984 | 10.792 | 0.235 |
| Volume; d | uL | 49.533 | 15.281 | 54.152 | 13.791 | 0.457 |
| Stroke Volume | uL | 27.240 | 12.723 | 27.168 | 8.530 | 0.988 |
| Ejection Fraction | % | 53.897 | 15.345 | 51.019 | 12.806 | 0.632 |
| Fractional Shortening | % | 27.990 | 9.549 | 25.859 | 7.672 | 0.564 |
| Cardiac Output | mL/min | 12.022 | 7.020 | 12.363 | 4.209 | 0.525 |
| LV Mass | mg | 108.525 | 27.250 | 104.428 | 16.534 | 0.671 |
| LV Mass Corrected | mg | 86.820 | 21.800 | 83.542 | 13.227 | 0.671 |
| IVS; s | mm | 1.343 | 0.338 | 1.160 | 0.152 | 0.116 |
| IVS; d | mm | 0.933 | 0.218 | 0.778 | 0.0994 | 0.043 |
| LVPW; s | mm | 1.163 | 0.270 | 1.124 | 0.145 | 0.406 |
| LVPW; d | mm | 0.866 | 0.175 | 0.844 | 0.132 | 0.538 |

MV

| | | | | | | |
|---|---|---|---|---|---|---|
| ET | ms | 51.329 | 6.226 | 45.749 | 5.814 | 0.038 |
| IVCT | ms | 19.946 | 8.573 | 17.184 | 3.744 | 0.336 |
| IVRT | ms | 25.210 | 12.960 | 26.001 | 4.264 | 0.844 |
| MV Deceleration | mm/s2 | 25325.958 | 12555.954 | 29694.529 | 5117.527 | 0.295 |
| MV Deceleration | ms | 22.958 | 5.589 | 19.243 | 3.932 | 0.082 |
| MV E/A | Ratio | 1.495 | 1.080 | 1.328 | 0.194 | 0.487 |

TDV

| | | | | | | |
|---|---|---|---|---|---|---|
| IVCT | ms | 20.456 | 4.778 | 18.735 | 3.256 | 0.342 |
| IVRT | ms | 18.430 | 1.533 | 22.484 | 4.301 | 0.035 |
| E/E' | Ratio | 41.195 | 17.342 | 41.862 | 13.293 | 0.730 |

PA

| | | | | | | |
|---|---|---|---|---|---|---|
| RVOT VTI | mmHg | 0.943 | 0.662 | 0.998 | 0.749 | 1.000 |

LA

| Length | mm | 1.830 | 0.189 | 1.698 | 0.133 | 0.078 |
|---|---|---|---|---|---|---|
| Mean LA Pressure | mmHg | 53.394 | 21.677 | 54.228 | 16.616 | 0.710 |
| 1 Month | | IgG | | TOMA | | |
| SAX | | AVG | SD | AVG | SD | P |
| Heart Rate | BPM | 446.238 | 170.000 | 347.496 | 57.087 | 0.069 |
| LVEDs/TL | Ratio | 0.136 | 0.0241 | 0.127 | 0.0211 | 0.327 |
| LVEDd/TL | Ratio | 0.190 | 0.0222 | 0.193 | 0.0213 | 0.770 |
| Volume; s | UL | 22.852 | 9.336 | 18.906 | 7.680 | 0.270 |
| Volume; d | UL | 50.043 | 13.152 | 51.183 | 11.918 | 0.826 |
| Stroke Volume | UL | 27.191 | 7.945 | 32.277 | 8.274 | 0.139 |
| Ejection Fraction | % | 55.333 | 10.998 | 63.349 | 9.527 | 0.069 |
| Fractional Shortening | % | 28.521 | 7.319 | 34.155 | 7.839 | 0.101 |
| Cardiac Output | mL/min | 11.751 | 3.957 | 11.026 | 2.681 | 0.604 |
| LV Mass | mg | 98.790 | 20.856 | 100.483 | 19.216 | 0.838 |
| LV Mass Corrected | mg | 79.032 | 16.685 | 80.386 | 15.373 | 0.838 |
| LVAW; s | mm | 1.201 | 0.238 | 1.261 | 0.211 | 0.518 |
| LVAW; d | mm | 0.877 | 0.133 | 0.826 | 0.127 | 0.347 |
| LVPW; s | mm | 1.158 | 0.135 | 1.225 | 0.199 | 0.346 |
| LVPW; d | mm | 0.854 | 0.0996 | 0.838 | 0.132 | 0.741 |

MV

| | | | | | | |
|---|---|---|---|---|---|---|
| AET | ms | 48.492 | 6.873 | 55.884 | 10.115 | 0.048 |
| IVCT | ms | 18.832 | 6.319 | 21.091 | 5.256 | 0.351 |
| IVRT | ms | 23.554 | 10.097 | 25.647 | 7.837 | 0.576 |
| MV Deceleration | ms | 21.865 | 3.728 | 25.925 | 4.954 | 0.034 |
| MV E/A | Ratio | 1.522 | 0.453 | 1.363 | 0.320 | 0.332 |

TDV

| | | | | | | |
|---|---|---|---|---|---|---|
| IVCT | ms | 20.229 | 4.529 | 23.197 | 4.790 | 0.143 |
| IVRT | ms | 15.140 | 3.187 | 20.135 | 6.329 | 0.042 |
| E/E' | Ratio | 44.638 | 22.677 | 38.639 | 10.837 | 0.421 |
| RVOT VTI | mmHg | 0.972 | 0.912 | 0.756 | 0.276 | 0.740 |

LA

| | | | | | | |
|---|---|---|---|---|---|---|
| Dimensions | mm | 2.019 | 0.207 | 1.735 | 0.237 | 0.011 |
| Mean LA Pressure | mmHg | 57.698 | 28.346 | 50.199 | 13.546 | 0.421 |

Abbreviations: SAX = M Mode short axis measurement;
LVEDd/TL = Left Ventricular (LV) End Diastolic Dimension in diastole corrected for tibia length;
LVEDs/TL = Left Ventricular End Diastolic Dimension in systole corrected for tibia length;
LVAW; s = Left ventricular anterior wall thickness in systole;
LVAW; d = Left ventricular anterior wall thickness in diastole;
LVPW; s = Left ventricular posterior wall thickness in systole;
LVPW; d = Left ventricular posterior wall thickness in diastole;
MV-mitral valve;
AET = Aortic Ejection Time;
IVCT = isovolumetric contraction time;
IVRT = isovolumetric relaxation time;
MV E/A = Mitral Valve (MV) ratio of E = blood flow peak velocity from left ventricular relaxation in early diastole to A = atrial contraction flow peak velocity in late diastole;
TDV = Tissue Doppler Velocity;
E/e' = early mitral inflow velocity to mitral annular early diastolic velocity ratio;
RVOT VTI = Right Ventricular outflow tract velocity time integral;
LA = Left Atrium.
hTau mice: 6 male 6 female injected with IgG control and 6 male 6 female injected with TOMA.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,816,567
U.S. Pat. No. 4,867,973
U.S. Pat. No. 4,938,948
U.S. Pat. No. 5,021,236
U.S. Pat. No. 5,141,648
U.S. Pat. No. 5,196,066
U.S. Pat. No. 5,563,250
U.S. Pat. No. 5,565,332
U.S. Pat. No. 5,856,456
U.S. Pat. No. 5,880,270
U.S. Pat. No. 6,485,982
“Antibodies: A Laboratory Manual,” Cold Spring Harbor Press, Cold Spring Harbor, N Y, 1988.
Abbondanzo et al., Am. J. Pediatr. Hematol. Oncol. 12(4): 480-489, 1990.
Allred et al., Arch. Surg. 125(1), 107-113: 1990.
Atherton et al., Biol. of Reproduction 32: 155-171, 1985.
Barzon et al., Euro Surveill. 11: 21(32), 2016.
Beltramello et al., Cell Host Microbe 8: 271-283, 2010.
Brown et al., J. Immunol. Meth. 130(1): 111-121, 1990.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Capaldi et al., Biochem. Biophys. Res. Comm. 74(2):4 25-433, 1977.
De Jager et al., Semin. Nucl. Med. 23(2): 165-179, 1993.
Dholakia et al., J Biol. Chem. 264: 20638-20642, 1989.
Diamond et al., J Virol. 77: 2578-2586, 2003.
Doolittle and Ben-Zeev, Methods Mol. Biol. 109: 215-237, 1999.
Duffy et al., N. Engl. J Med. 360: 2536-2543, 2009.
Elder et al. Infections, infertility and assisted reproduction. Part II: Infections in reproductive medicine & Part III: Infections and the assisted reproductive laboratory. Cambridge UK: Cambridge University Press; 2005.
Gefter et al., Somatic Cell Genet. 3: 231-236, 1977.
Gornet et al., Semin Reprod Med. 34(5): 285-292, 2016.
Gulbis and Galand, Hum. Pathol. 24(12): 1271-1285, 1993.
Halfon et al., PLoS ONE 5(5): e10569, 2010
Hessell et al., Nature 449: 101-104, 2007.
Khatoon et al., Ann. of Neurology 26: 210-219, 1989.
King et al., J. Biol. Chem. 269: 10210-10218, 1989.
Kohler and Milstein, Eur. J. Immunol. 6: 511-519, 1976.
Kohler and Milstein, Nature 256: 495-497, 1975.
Kyte and Doolittle, J. Mol. Biol. 157(1): 105-132, 1982.
Mansuy et al., Lancet Infect Dis. 16(10): 1106-7, 2016.
Nakamura et al., In: Enzyme Immunoassays: Heterogeneous and Homogeneous Systems, Chapter 27, 1987.
O'Shannessy et al., J. Immun. Meth. 99: 153-161, 1987.
Persic et al., Gene 187: 1, 1997
Potter and Haley, Meth. Enzymol. 91: 613-633, 1983.
Purpura et al., Lancet Infect Dis. 16(10): 1107-8, 2016.
Remington's Pharmaceutical Sciences, 15th Ed., 3: 624-652, 1990.
Tang et al., J. Biol. Chem., 271: 28324-28330, 1996.
Wawrzynczak & Thorpe, In: Immunoconjugates, Antibody Conjugates In Radioimaging And Therapy Of Cancer, Vogel (Ed.), NY, Oxford University Press, 28, 1987.
Yu et al., J Immunol Methods 336: 142-151, 2008.
Ballatore et al., Nat Rev Neurosci 2007; 8(9):663-72.
Kovacs G. Tauopathies. Handbook of Clinical Neurology 2018; 145:355-368.
Iadecola C. Nat Rev Neurosci 2004; 5(5):347-60.
Ruitenberg et al., Ann Neurol 2005; 57(6):789-94.
Iturria-Medina et al., Nat Commun 2016; 7:11934.
Diteepeng et al., Eur J Clin Invest 2021:e13504.
Daniele et al., Heart and Brain: Complex Relationships for Left Ventricular Dysfunction. Curr Cardiol Rep 2020; 22(8):72.
Troncone et al., J Am Coll Cardiol 2016; 68(22):2395-2407.
Drastichova et al., PLoS One 2012; 7(10):e47167.
Georgieff et al., J Cell Sci 1993; 105 (Pt 3):729-37.
Bers D. Excitation-Contraction Coupling and Cardiac Contractile Force. Dordrecht, the Netherlands: Kluwer Academic Publishers 2001(2nd ed.).
Bers D M. Circ Res 2000; 87(4):275-81.
del Monte et al., J Card Fail 2002; 8(6 Suppl):S421-31.
Sevigny et al., Nature 2016; 537(7618):50-6.
Bernini et al., Sci Rep 2016; 6:35865.
Castillo-Carranza et al., J Neurosci 2015; 35(12):4857-68.
Sjogren et al., J Neurol Neurosurg Psychiatry 2001; 70(5):624-30.
Taylor et al., Circulation 2007; 115(10):1244-51.
Augustinack Acta Neuropathol 2002; 103(1):26-35.
Wille et al., J Cell Biol 1992; 118(3):573-84.
Schneider Biochemistry 1999; 38(12):3549-58.
Andorfer et al., J Neurochem 2003; 86(3):582-90.
Polydoro J Neurosci 2009; 29(34):10741-9.
Geiszler et al., Neuroscience 2016; 329:98-111.
Phillips et al., PLoS One 2011; 6(5):e19567.
Andorfer et al., J Neurosci 2005; 25(22):5446-54.
Chen et al., Neurobiol Dis 2015; 76:13-23.
Chatrchyan et al., Physical review letters 2012; 109(2):022301.
Demuro A, Parker I. J Neurosci 2013; 33(9):3824-33.
Demuro et al., J Biol Chem 2010; 285(17):12463-8.
Esteras et al., FEBS J 2021; 288(1):127-141.
Gianni et al., Circulation 2010; 121(10):1216-26.
Castillo-Carranza et al., J Neurosci 2014; 34(12):4260-72.
Wang et al., Nat Rev Neurol 2017; 13(10):612-623.
Kuo et al., Am J Pathol 2000; 156(3):797-805.
Joachim et al., Nature 1989; 341(6239):226-30.
Akerman et al., J Alzheimers Dis 2019; 69(2):463-478.
Sanbe et al., Proc Natl Acad Sci USA 2004; 101(27):10132-6.
Subramanian et al., J Am Coll Cardiol 2015; 65(12):1199-1214.
Betrie et al., J Alzheimers Dis 2017; 56(2):849-860.
Tagawa et al., Circ Res 1998; 82(7):751-61.
Sotiropoulos et al., Acta Neuropathol Commun 2017; 5(1):91.
Drubin et al., J Cell Biol 1988; 106(5):1583-91.
Boyne et al., J Comp Neurol 1995; 358(2):279-93.
Nothias et al., Neuroscience 1995; 66(3):707-19.
Fischer I, Baas P W. Trends Neurosci 2020; 43(7):493-504.
Kosik K S. Brain Pathol 1993; 3(1):39-43.
Avila J. FEBS Lett 2000; 476(1-2):89-92.
Iijima-Ando et al., Brain Struct Funct 2010; 214(2-3):245-62.
Majd et al., IBRO Rep 2017; 2:1-13.
Despres et al., Proc Natl Acad Sci USA 2017; 114(34):9080-9085.
Saad N S, Elnakish M T, Ahmed A A E, Janssen P M L. Protein Kinase A as a Promising Target for Heart Failure Drug Development. Arch Med Res 2018; 49(8):530-537.
Schiattarella et al., Circulation 2020; 141(9):709-711.
Sanderson J E. Eur Heart J Qual Care Clin Outcomes 2016; 2(3):153-163.
Shafiei et al., Front Aging Neurosci 2017; 9:83.
Kolstad et al., Elife 2018; 7.
Ponikowski et al., ESC Heart Fail 2014; 1(1):4-25.
2020 Alzheimer's disease facts and figures. Alzheimers Dement 2020.
Van Der Lee, S., Acta Physiol. Pharmacol. Neerl 4 (3):442-444, 1955.
Van Der Lee S., Acta Physiol. Pharmacol. Neerl 5 (2): 213-215, 1956.
Whitten W. K., J. Endocrinol. 18 (1) 102-107, 1959.
Gerson J. E., Mol. Neurodegener. 13 (1) 13, 2018.
Graham E. L., J. Vis. Exp. 79, e50289, 2013.
Del Monte, F., Circulation 100 (23); 2308-2311, 1999.
Castillo-Carranza, D. L., J. Neurosci. 34(12): 4260-72., 2014.
Lo Cascio, F., J. Biol. Chem. 295(44): 14807-14825, 2020.
Sengupta, U. C-MM., Kayed, R., Methods in Molecular Biology (Clifton, N. J.) 1779:113, 2017.
Castillo-Carranza, D. L., Aging Dis. 8(3):257-266, 2017.
Montalbano et al., Nat. Commun., 11(1):4305, 2020.
Albayram et al., Nat. Commun. 8(1):1000, 2017.
Albayram et al., Sci. Rep. 9(1):8646, 2019.
Castillo-Carranza, D. L., J. Alz. Dis. 40 (suppl1): S97-S111, 2014.

1. A method of detecting a misfolded protein in the heart tissue of a subject comprising:
(a) contacting a heart tissue sample from said subject with an antibody or antibody fragment that binds to Cofilin, Aβ or Tau; and
(b) detecting Cofilin, Aβ or Tau in said sample by binding of said antibody or antibody fragment to a Cofilin, Aβ or Tau in said sample.

2. The method of claim 1, wherein said sample may be a tissue sample or a body fluid.

3. The method of claim 1, wherein said tissue sample may be a cardiac tissue sample.

4. The method of claim 1, wherein detection comprises ELISA, RIA, lateral flow assay or Western blot.

5. The method of claim 1, further comprising performing steps (a) and (b) a second time and determining a change in Cofilin, Aβ or Tau levels as compared to the first assay.

6-8. (canceled)

9. The method of claim 1, wherein the antibody fragment is a recombinant scFv (single chain fragment variable) antibody, Fab fragment, F(ab′)₂ fragment, or Fv fragment.

10. A method of treating a subject with heart disease characterized by misfolded proteins in cardiac tissue, or reducing the likelihood of the same, comprising delivering to said subject an antibody or antibody fragment that binds to Cofilin, Aβ or Tau.

11-13. (canceled)

14. The method of claim 1, wherein the antibody fragment is a recombinant scFv (single chain fragment variable) antibody, Fab fragment, F(ab′)₂ fragment, or Fv fragment.

15. The method of claim 10, wherein said antibody is an IgG, or a recombinant IgG antibody or antibody fragment comprising an Fc portion mutated to alter (eliminate or enhance) FcR interactions, to increase half-life and/or increase therapeutic efficacy, such as a LALA, LALA-PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern.

16. The method of claim 10, wherein said antibody is a chimeric antibody or a bispecific antibody.

17. The method of claim 10, wherein said antibody or antibody fragment is administered multiple times.

18. (canceled)

19. The method of claim 10, wherein delivering comprises antibody or antibody fragment administration, or genetic delivery with an RNA or DNA sequence or vector encoding the antibody or antibody fragment.

20. A vaccine formulation comprising one or more antibodies or antibody fragments that bind to Cofilin, Aβ and/or Tau.

21-24. (canceled)

25. The vaccine formulation of claim 20, further comprising a second heart failure therapy.

26. The vaccine formulation of claim 20, wherein at least one of said antibody fragments is a recombinant scFv (single chain fragment variable) antibody, Fab fragment, F(ab′)₂ fragment, or Fv fragment.

27. The vaccine formulation of claim 20, wherein at least one of said antibodies is a chimeric antibody or is bispecific antibody.

28. The vaccine formulation of claim 20, wherein at least one of said antibodies is an IgG, or a recombinant IgG antibody or antibody fragment comprising an Fc portion mutated to alter (eliminate or enhance) FcR interactions, to increase half-life and/or increase therapeutic efficacy, such as a LALA, LALA-PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern.

29. The vaccine formulation of claim 20, wherein at least one of said antibodies or antibody fragments further comprises a cell penetrating peptide and/or is an intrabody.

30. A vaccine formulation comprising one or more expression vectors encoding a first antibody or antibody fragment that binds to Cofilin, Aβ or Tau.

31. The vaccine formulation of claim 30, wherein said expression vector(s) is/are Sindbis virus or VEE vector(s).

32. The vaccine formulation of claim 30, formulated for delivery by needle injection, jet injection, or electroporation.

33. The vaccine formulation of claim 30, further comprising one or more expression vectors encoding for a second antibody or antibody fragment that binds to Cofilin, Aβ or Tau, such as wherein the second antibody binds to a different protein than the first antibody.

34. The vaccine formulation of claim 30, wherein the formulation comprises one or more expression vectors that encoding and antibodies or antibody fragments that bind to Cofilin, Aβ and Tau.
